(19) United States
(12) Patent Application Publication
(10) Pub. No.: US 2025/0205328 A1
(43) Pub. Date: Jun. 26, 2025

(54) CONTROLLED RELEASE VACCINE FORMULATIONS

(71) Applicant: Merck Sharp & Dohme LLC, Rahway, NJ (US)

(72) Inventors: Akhilesh Bhambhani, Foster City, CA (US); Donna M. Williams, Glenside, PA (US)

(73) Assignee: Merck Sharp & Dohme LLC, Rahway, NJ (US)

(21) Appl. No.: 18/847,437

(22) PCT Filed: Mar. 23, 2023
(86) PCT No.: PCT/US2023/016019

§ 371 Date: Sep. 16, 2024

Related U.S. Application Data
(60) Provisional application No. 63/323,903, filed on Mar. 25, 2022.

(51) Int. Cl. A61K 39/12; A61K 9/00; A61K 9/20; A61K 9/28; A61K 9/51; A61K 39/00; A61P 31/20; C12N 7/00

(52) U.S. Cl. CPC A61K 39/12; A61K 9/0024; A61K 9/2018; A61K 9/2853; A61K 9/5153; A61P 31/20; C12N 7/00; A61K 2039/5258; A61K 2039/54; A61K 2039/545; A61K 2039/55505; A61K 2039/55555; C12N 2710/20023; C12N 2710/20034

(57) ABSTRACT

The present disclosure provides, among other things, a vaccine composition that includes HPV virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, where the vaccine composition provides enhanced or comparable HPV vaccine response in comparison to a similar multiple-dose vaccine.

### CROSS-REFERENCE TO RELATED APPLICATIONS

This application claims the benefit of U.S. Provisional Patent Application Ser. No. 63/323,903 filed Mar. 25, 2022, the entire contents of which are incorporated by reference herein.

### FIELD

The present invention relates generally to the prevention of human papillomavirus (HPV) infection. More specifically, the invention relates to pharmaceutical compositions and formulations comprising virus-like particles (VLPs) of HPV and PLGA, which can be administered as a single-dose vaccine. The present disclosure provides, among other things, a single-dose vaccine composition that includes an HPV vaccine, where a single administration of the vaccine composition provides a comparable or enhanced immune response in comparison to multiple administrations of the same HPV vaccine formulated. Further provided are methods of using the disclosed compositions and formulations.

### BACKGROUND

Human papillomaviruses (HPVs) are small, double-stranded DNA viruses that infect the skin and internal squamous mucosal epithelia of men and women. HPVs are classified based on their carcinogenic properties. HPVs include major (L1) and minor (L2) capsid proteins. Over 200 distinct HPV genotypes have been identified (Li et al., “Rational design of a triple-type human papillomavirus vaccine by compromising viral-type specificity,” Nature, 9:5360 (2018)), many of which have been associated with pathologies ranging from benign proliferative warts to malignant carcinomas of the cervix (for review, see McMurray et al., Int. J. Exp. Pathol. 82(1): 15-33 (2001)). Those HPV types labeled as “high-risk” include 16, 18, 31, 33, 35, 39, 45, 51, 52, 56, 58, 68, and 59. (Chan et al., “Human Papillomavirus Infection and Cervical Cancer: Epidemiology, Screening, and Vaccination—Review of Current Perspectives,” Journal of Oncology, vol. 2019, Article ID 3257939, 2019.)

HPV is the primary etiological agent in cervical cancer, one of the most common cancer types in women, as well as squamous cell carcinomas of the anus, tonsil, tongue, vulva, vagina, and penis. HPV16 and HPV18 are well known as the most virulent of the high-risk HPV types as they cause approximately 70% of all invasive cervical cancer in the world.

Papillomaviruses are small (50-60 run), nonenveloped, icosahedral DNA viruses that encode up to eight early (E1-E7) and two late (L1-L2) genes. The L1 protein is the major capsid protein and has a molecular weight of 55-60 kDa. Expression of the L1 protein or a combination of the L1 and L2 proteins in yeast, insect cells, mammalian cells or bacteria leads to self-assembly of virus-like particles (VLPs) (for review, see Schiller and Roden, in Papillomavirus Reviews: Current Research on Papillomaviruses; Lacey, ed. Leeds, UK: Leeds Medical Information, pp 101-12 (1996)).

VLPs are morphologically similar to authentic virions and are capable of inducing high titers of neutralizing antibodies upon administration into animals or humans. Because VLPs do not contain the potentially oncogenic viral genome, they present a safe alternative to the use of live virus in HPV vaccine development (for review, see Schiller and Hidesheim, J Clin. Virol. 19: 67-74 (2000)). For this reason, the L1 and L2 genes have been identified as immunological targets for the development of prophylactic and therapeutic vaccines for HPV infection and disease.

VLP-based vaccines have proven to be effective at inducing immune responses in human subjects vaccinated with bivalent HPV 16 and 18 (Harper et al. Lancet 364 (9447): 1757-65 (2004)), quadrivalent HPV 6, 11, 16, and 18 (Villa et al. Vaccine 24: 5571-5583 (2006)) and multi-valent HPV 6, 11, 16, 18, 31, 33, 45, 52 and 58 VLP-based vaccines. Three marketed VLP-based vaccines against HPV are administered according to 2 or 3 dose regimens. CERVARIX® (GlaxoSmithKline Biologicals, Rixensart, Belgium), is a bivalent vaccine protective against HPV 16 and 18. GARDASIL® and GARDASIL®9 (Merck & Co., Inc., Rahway, NJ, USA) protect against two and seven additional HPV types, respectively, and prevent additional HPV-related anogenital diseases, including wart formation. The additional five high-risk strains in GARDASIL®9 compared to GARDASIL® increases protection from about 70% of anogenital malignancies to about 90%. (Id., M. Nygard, et al., “Evaluation of the long-term anti-human papillomavirus 6 (HPV6), 11, 16, and 18 immune responses generated by the quadrivalent HPV vaccine,” Clinical and Vaccine Immunology, vol. 22, no. 8, pp. 943-948, 2015.)

### SUMMARY

The present disclosure provides a pharmaceutical composition comprising: virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82; a polymer comprising poly(lactide-co-glycolide); and a pharmaceutically acceptable carrier.

In one aspect, the present disclosure provides a pharmaceutical composition suitable for parenteral administration, comprising: (a) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82; (b) a polymer comprising poly(lactide-co-glycolide); and (c) a pharmaceutically acceptable carrier, wherein the weight content of the HVP vaccine component in the composition is within a range from 0.01% to 20%; the weight content of the polymer in the pharmaceutical composition is within a range from 80% to 99.09%; and wherein the polymer has an intrinsic viscosity of 0.4-0.9 dl/g, and a molar ratio of lactide to glycolide of 65:35 to 90:10.

In another aspect, the present disclosure provides a kit comprising: (a) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82; a poly(lactide-co-glycolide); and a pharmaceutically acceptable carrier; and (b) a delivery device.

In another aspect, the present disclosure provides a pulsatile drug delivery system comprising: a microneedle assembly including a plurality of microneedles filled with a composition comprising: virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82; poly(lactide-co-glycolide); and a pharmaceutically acceptable carrier, wherein the microneedle assembly is configured to release the VLPs at predetermined times with a predetermined amount of the VLPs while the microneedle assembly remains embedded in a patient.

The summary of the technology described above is non-limiting and other features and advantages of the technology will be apparent from the following detailed description, and from the claims.

### BRIEF DESCRIPTION OF THE DRAWINGS

FIG. 1A depicts a representation of a formulation of the present invention that includes a core and shell polymer. FIG. 1B depicts a graphical representation of the formulation when released as a liquid and burst overtime.

FIG. 2A shows a core-shell microsphere of the present invention. FIG. 2B shows a sub dermal implant of the present invention. FIG. 2C shows a core-shell lyosphere of the present invention. FIG. 2D shows a microparticle of the present invention.

FIG. 3 is an SEM image of ProCept spray dried materials. See, e.g. Example 1.

FIG. 4A is a graphical comparison of Buchi vs ProCept HPV VLP potency. FIG. 4B is a graphical comparison of Buchi vs ProCept AAHS particle size. See, e.g. Example 1.

FIG. 5 is a graphical representation of a scheme of the present invention. See, e.g. Example 2.

FIG. 6. is a graphical representation of HPV potency for different formulations after microparticle encapsulation. See, e.g. Example 2.

FIG. 7A is a graphical depiction of ELISA titers of various formulations. FIG. 7B is a graphical representation of ELISA titers of various formulations over 120 days post vaccination. See, e.g. Example 2.

FIG. 8 is a graphical depiction of a scheme for forming microparticles of the present invention. See, e.g. Example 2.

FIG. 9A is a graphical depiction of an embodiment of the present invention to show the antibody response of VLP HPV16 over 80 days. FIG. 9B is a graphical depiction of an embodiment of the present invention to show the antibody response of VLP HPV18 over 80 days. See e.g. Example 2.

FIG. 10 is a graphical depiction of the potency of various embodiments of the present invention. See, e.g. Example 3.

FIG. 11A is a graphical depiction of the potency of HPV minitabs that do not include AAHS. FIG. 11B is a graphical depiction of the potency of HPV minitabs that do include AAHS. See, e.g. Example 3.

FIGS. 12A-12B are a graphical depictions of the immunogenicity evaluation of various formulations of the present invention. See, e.g. Example 3.

FIG. 13 depicts the graphical representation of the relative potency of various embodiments of the present invention. See, e.g. Example 4.

Various aspects described herein are described in further detail in the following subsections.

### DETAILED DESCRIPTION

Currently, there are multiple approved HPV vaccines that are composed of engineered virus like particles (VLPs) and are highly effective at protecting vaccinated patients against premalignant lesions and anogenital cancers and genital warts when administered prior to natural infection in subjects 9 years and older as multidose regimens. The compositions of the present invention are intended to generate immunity against HPV subtypes through a single-administration regimen that is comparable to a 2-3 injection regimen of such HPV vaccine, including an approved two, four, or nine valent HPV vaccine.

Though improving, worldwide HPV vaccination rates remain suboptimal. The worldwide coverage of HPV vaccination rates can be improved by reducing the number of healthcare practitioner visits required for the vaccination, increasing education on HPV disease prophylaxis, and alleviating the social stigma associated with vaccination. The proportion of adolescents in the Americas and in Europe completing a two dose vaccination series is estimated to be under 50%. Accordingly, it is desirable to improve HPV vaccination rates by generating improved vaccines that can generate immunity against HPV through a single administration that provides a comparable immune response to existing HPV vaccines that require 2 or more doses.

It is desirable to provide a vaccine that can be delivered alone or in combination with a solution/suspension in one doctor's visit to greatly improve the number of individuals that will be protected from HPV. Accordingly, the present disclosure is directed to improved methods of delivering an HPV vaccine to an individual in need thereof. In some embodiments, the present disclosure provides a method of stabilizing HPV that is either bound to aluminum hydroxyphosphate sulfate adjuvant (“AAHS”) or unbound to AAHS. In another embodiment, the present disclosure provides a process of making a spray dried HPV-AAHS formulations

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” includes the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

AAHS: As used herein, the term “AAHS” refers to an amorphous aluminum hydroxyphosphate sulfate adjuvant.

About: As used herein, the term “about,” when used herein in reference to a value, refers to a value that is the same as or, in context, is similar to the referenced value. In general, those skilled in the art, familiar with the context, will appreciate the absolute amount and/or relative degree of difference encompassed by “about” in that context. As used herein, the term “about” in quantitative terms refers to plus or minus 10% of the value it modifies (rounded up to the nearest whole number if the value is not sub-dividable, such as a number of molecules or nucleotides). For example, in some embodiments, the term “about” can encompass a range of values that within 25%, 20%, 19%, 18%, 17%, 16%, 15%, 14%, 13%, 12%, 1%, 10%, 9%, 8%, 7%, 6%, 5%, 4%, 3%, 2%, 1%, or less of the referenced value.

Adjuvant: As used herein, the term “adjuvant” refers to a composition or compound that is capable of enhancing the immune response against an antigen of interest. Adjuvants are substances or combinations of substances that are used in conjunction with a vaccine antigen to enhance (e.g., increase, accelerate, prolong and/or possibly target) the specific immune response to the vaccine antigen or modulate to a different type (e.g., switch a Th1 immune response to a Th2 response, or a humoral response to a cytotoxic T cell response) in order to enhance the clinical effectiveness of the vaccine. In some embodiments, the adjuvant modifies (Th1/Th2) the immune response. In some embodiments, the adjuvant boosts the strength and longevity of the immune response. In some embodiments, the adjuvant broadens the immune response to a concomitantly administered antigen. In some embodiments, the adjuvant is capable of inducing strong antibody and T cell responses. In some embodiments, the adjuvant is capable of increasing the polyclonal ability of the induced antibodies. In some embodiments, the adjuvant is used to decrease the amount of antigen necessary to provoke the desired immune response and provide protection against the disease. In some embodiments, the adjuvant is used to decrease the number of injections needed in a clinical regimen to induce a durable immune response and provide protection against the disease. Adjuvant containing formulations described herein may demonstrate enhancements in humoral and/or cellular immunogenicity of vaccine antigens, for example, subunit vaccine antigens. Adjuvants of the present invention are not used to deliver antigens, antibodies, active pharmaceutical ingredients (APIs), or VLPs.

Administration: As used herein, the term “administration” refers to the act of providing an active agent, composition, or formulation to a subject. Exemplary routes of administration to the human body can be through the eyes (ophthalmic), mouth (oral), skin (transdermal), nose (nasal), lungs (inhalant), rectal, vaginal, oral mucosa (buccal), ear, by injection (e.g., intravenously (IV), subcutaneously, intratumorally, intraperitoneally, intramuscularly (IM), intradermally (ID) etc.) and the like.

Agent: As used herein, the term “agent” refers to a particle, compound, molecule, or entity of any chemical class including, for example, a VLP, a small molecule, polypeptide (e.g., a protein), polynucleotide (e.g., a DNA polynucleotide or an RNA polynucleotide), saccharide, lipid, or a combination or complex thereof. In some embodiments, the term “agent” can refer to a compound, molecule, or entity that includes a polymer, or a plurality thereof.

Antibody: As used herein, the term “antibody” (or “Ab”) refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies.

Antigen: As used herein, the term “antigen” refers to any antigen that can generate one or more immune responses. The antigen may be a protein (including recombinant proteins), VLP, polypeptide, or peptide (including synthetic peptides). The antigen may be one that generates a humoral and/or CTL immune response.

API: As used herein, the term “API” refers to an active pharmaceutical ingredient, e.g. HPV VLPs, which is a component of the compositions or formulations disclosed herein that is biologically active (e.g. capable of inducing an appropriate immune response) and confers a therapeutic or prophylactic benefit to a person or animal in need thereof. As used herein, an API is a vaccine active ingredient.

Biocompatible: As used herein, the term “biocompatible” refers to a material that is not toxic to the body, is pharmaceutically acceptable, is not carcinogenic.

Biodegradable: As used herein, the term “biodegradable” refers to a material that should degrade by bodily processes to products readily disposable by the body and should not accumulate in the body. The products of the biodegradation should also be biocompatible with the body.

Body: As used herein, “body” preferably refers to the human body, but it should be understood that body can also refer to a non-human animal body.

Controlled Release: As used herein, “controlled release” refers to the rate at which an active agent or other type of substance is released into the body as a function of time.

Dose: As used herein, the term “dose” means a quantity of an agent, API, formulation, or pharmaceutical composition administered or recommended to be administered at a particular time.

HPV and PV: As used herein, the terms “HPV” and “PV” refer to human papillomavirus and papillomavirus, respectively.

Microparticles: As used herein, the terms “microparticles” refers to particles that contain an active agent or other substance dispersed or dissolved within a polymer that serves as a matrix or binder of the particle.

Microspheres: As used herein, the term “microspheres” refers to spherical or spherical-like particles consist of drug dissolved and (or) dispersed homogeneously throughout a polymer material, with a particle size ranging in 1-500 μm, and generally prepared as suspensions for injection.

Multiple-dose: As used herein, the term “multiple-dose” refers to a vaccine composition, or pharmaceutical composition, that requires more than one dose of the components therein in a clinical regimen to induce a durable immune response and provide protection from a disease. One of skill in the art would understand how to determine a durable immune response, e.g., by measuring antibody titers over a specified period of time.

Patient: As used herein, the term “patient” refers to any human being that is to receive the HPV vaccines, or pharmaceutical compositions, described herein. As defined herein, “patient” includes those already infected with one or more types of HPV as well as those in which infection with one or more types of HPV is to be prevented.

Pharmaceutically acceptable: As used herein, the term “pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

Pharmaceutical composition: As used herein, the term “pharmaceutical composition,” refers to a composition containing an active pharmaceutical or biological ingredient, along with one or more additional components, e.g. a composition in which an active agent is formulated together with one or more pharmaceutically acceptable carriers. As used herein, the terms “pharmaceutical formulation” and “formulation” are used interchangeably with “pharmaceutical composition.” In some embodiments, the active agent is present in a unit dose amount appropriate for administration in a therapeutic regimen that shows a statistically significant probability of achieving a predetermined therapeutic effect when administered to a relevant population. The pharmaceutical compositions or formulations can be liquid or solid (e.g., lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. The pharmaceutical compositions or formulations are nontoxic to recipients at the dosages and concentrations employed. In some embodiments, a pharmaceutical composition can be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, the term formulation refers to a single-dose of vaccine, which can be included in any volume suitable for injection.

Single-dose: As used herein, the term “single-dose” refers to a vaccine composition that only requires one administration or injection in a clinical regimen to induce a durable immune response and provide protection from a disease. One of skill in the art would understand how to determine a durable immune response, e.g., by measuring antibody titers over a specified period of time.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Therapeutically Effective Amount: As used herein, the term “therapeutically effective amount” refers to an amount of the active ingredient (e.g. therapeutic protein, vaccine, or antibody) sufficient to produce the desired therapeutic effect in a human or animal, e.g. the amount necessary to elicit an immune response, treat, cure, prevent, or inhibit development and progression of a disease or the symptoms thereof and/or the amount necessary to ameliorate symptoms or cause regression of a disease. Therapeutically effective amount may vary depending on the structure and potency of the active ingredient and the contemplated mode of administration. One of skill in the art can readily determine a therapeutically effective amount of a given antibody or therapeutic protein or vaccine antigen.

Vaccine: As used herein, the term “vaccine” or “vaccine composition” refers to a substance used to stimulate the production of antibodies and provide immunity against one or several diseases, prepared from the causative agent of a disease, its products, or a synthetic substitute, treated to act as an antigen without inducing the disease. A vaccine composition may include at least one antigen or VLP in a pharmaceutically acceptable vehicle useful for inducing an immune response in a subject. The vaccine composition is administered by doses and techniques known to those skilled in the pharmaceutical or veterinary fields, taking into account factors such as the age, sex, weight, species, and condition of the recipient animal and the route of administration.

Valent: As used herein, the term “valent” refers to the presence of a specified number of antigens in a molecule. For example, the terms bi-valent, bivalent, 2 valent, or 2-valent refer to two different antigens. Similarly, the terms quadrivalent, 4 valent, or 4-valent refer to four different antigens and the terms nonavalent, 9 valent or 9-valent refer to nine different antigens.

Viscosity: As used herein, viscosity refers to the measure of a substance's resistance to deformation or flow at a given rate. Viscosity can be measured, for example, by using a viscometer at a given shear rate or shear rates that are appropriately selected by those skilled in the art to accurately measure viscosity in the viscosity range of the sample of interest. The viscosity of the substances used herein were measured using a viscometer (e.g. Brookfield DVII+pro) at 20° C. at a standard concentration (e.g. 1% in 1% acetic acid).

Virus Like Particles: As used herein, the term “virus like particles” or “VLPs” refers to agents that are morphologically similar to authentic virions or provide an arrayed display of an antigen and are capable of inducing high antibody neutralization ratings after administration in an animal. VLPs lack the viral genetic material of the authentic virions and are thus non-infectious.

Volume %, or % by volume: As used herein, the terms “volume %” or “% by volume” refers to parts by volume per hundred parts total volume of polymer carrier, e.g. microparticle, microsphere, or minitab. Unless otherwise indicated to the contrary, percentages (%) reported herein are by volume.

Weight % or % by weight: As used herein, the terms “weight %” or “% by weight” refers to parts by weight per hundred parts total weight of volume of polymer carrier, e.g. microparticle, microsphere, or minitab. For example, 10 wt. % active agent would mean 10 parts active agent by weight and 90 parts polymer by weight.

### Microparticles

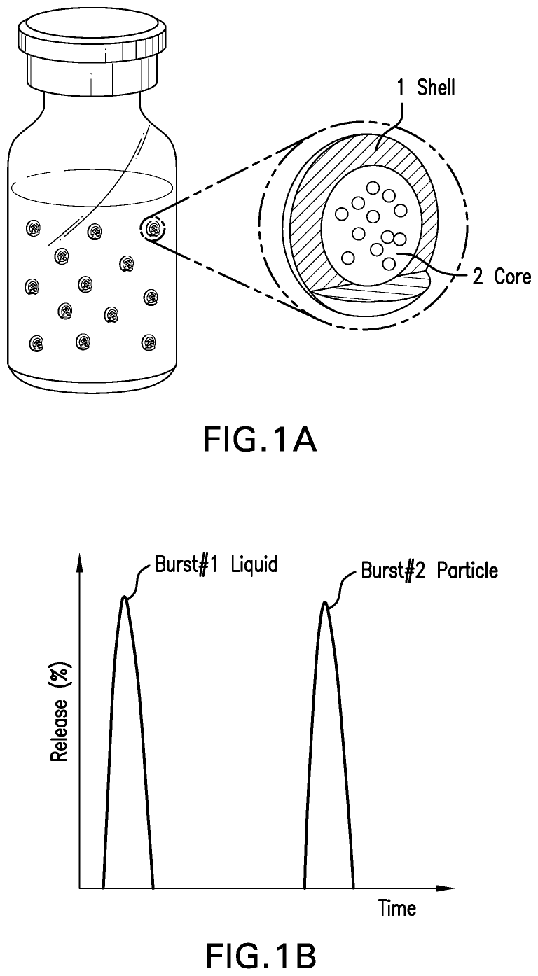

The present disclosure is also directed to anew way of delivering HPV bound to AAHS and unbound to AAHS via the temperature-programmed loading of HPV to make monolithic PLGA microparticles. See FIGS. 1A-1B. The present disclosure is also directed to a process to prepare a sterile sustained-release formulation of 9-valent Human Papillomavirus (HPV) vaccine with or without an adjuvant via temperature-programmed loading of poly(lactic-co-glycolic acid) (PLGA) microparticles for HPV unbound to AAHS. Encapsulating biomacromolecules in their native state within polymeric matrix remains challenging as harsh processing conditions are usually involved in traditional microencapsulation techniques (e.g., solvent evaporation, coacervation, spray drying, and microfluidics). The process described here significantly minimized the impact from common factors attributing to protein instability during microencapsulation, such as organic solvent exposure, excessive heating, or mechanical stress conditions. In the case HPV was bound to AAHS, monolithic PLGA microparticles were prepared by encapsulating HPV/AAHS via double emulsion process.

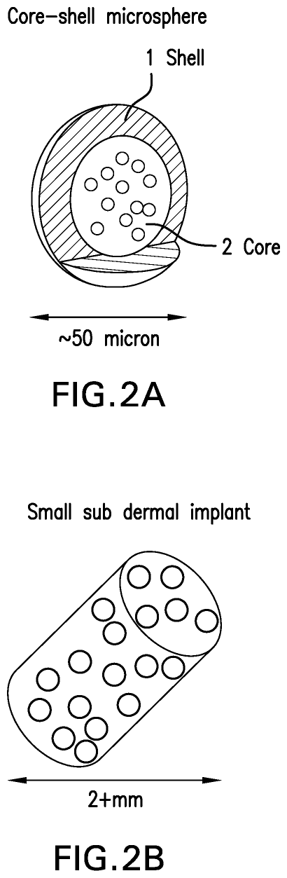

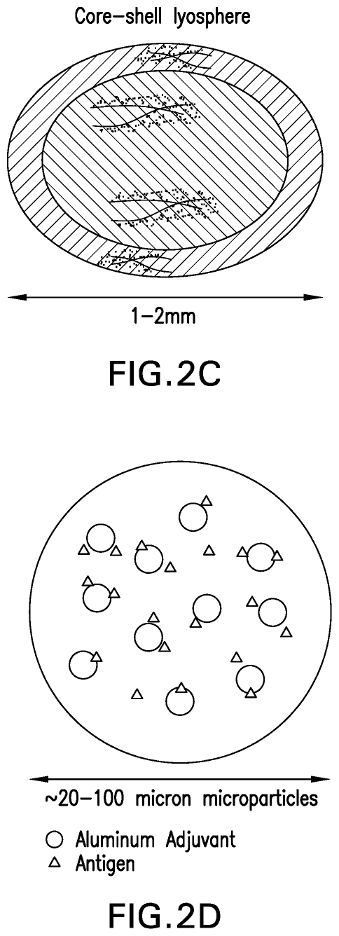

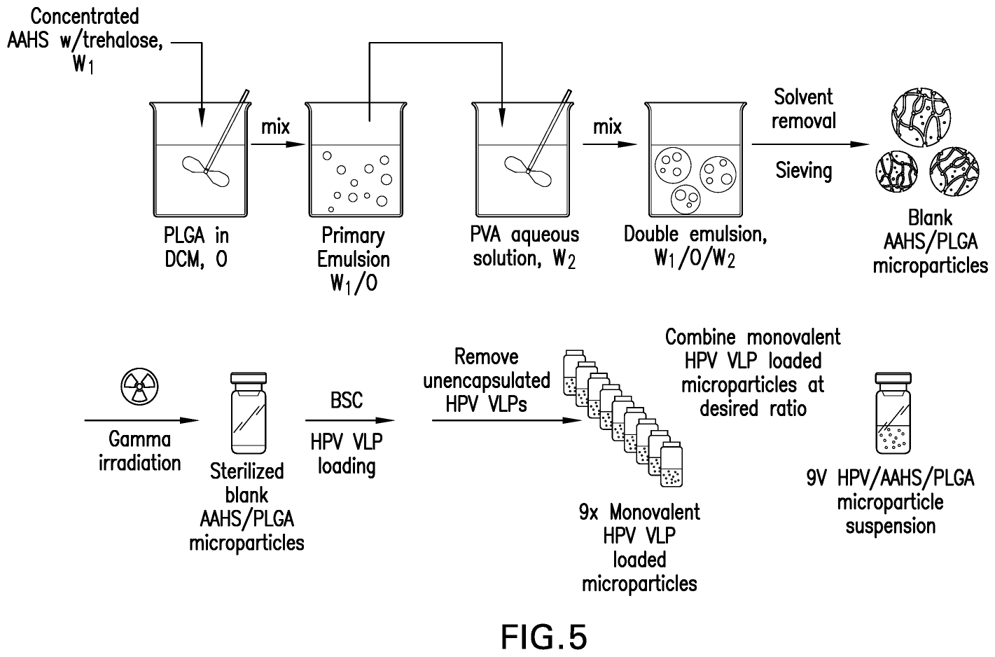

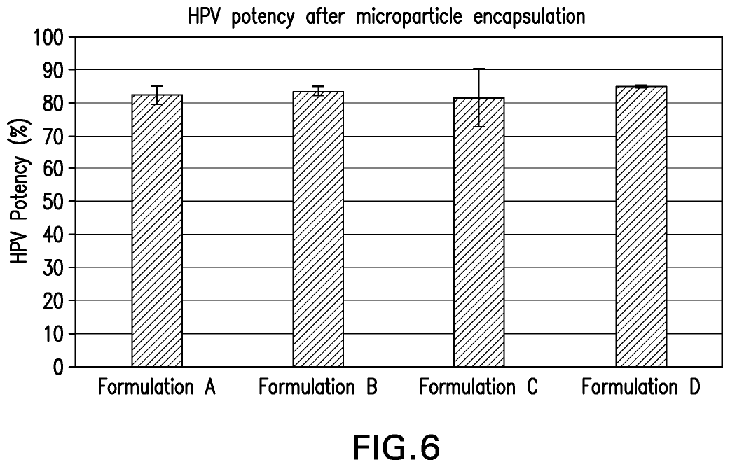

In some embodiments, the HPV vaccine is delivered via microparticles. See FIGS. 2C and 2D. Microparticles of the invention may include a polymer. In some embodiments, the polymer is a lactide-glycolide copolymer, also referred to as also referred to as poly(lactide-co-glycolide) and commonly abbreviated as PLGA (“PLGA”). See FIGS. 5 and 6.

In some embodiments, the PLGA is an “uncapped poly(lactide-co-glycolide),” which refers to poly(lactide-co-glycolide) having a carboxyl terminal group (“uncapped PLGA”). In some embodiments, the PLGA is a “capped poly(lactide-co-glycolide),” which refers to poly(lactide-co-glycolide) having an ester group (“capped PLGA”).

The two copolymers, i.e., the two PLGAs, are a capped PLGA with a high intrinsic viscosity of 0.4-0.9 dl/g, preferably, 0.45-0.8 dl/g, more preferably, 0.45-0.55 dl/g, and a capped PLGA with a low intrinsic viscosity of 0.1-0.45 dl/g, preferably, 0.1-0.3 dl/g, more preferably, 0.2-0.3 dl/g. In some embodiments, the weight ratio of the capped PLGA with the high intrinsic viscosity to the capped PLGA with the low intrinsic viscosity is (100:0):(50:50). In some embodiments, the weight ratio of the capped PLGA with the high intrinsic viscosity to the capped PLGA with the low intrinsic viscosity is (50-95):(5-50). In some embodiments, the weight ratio of the capped PLGA with the high intrinsic viscosity to the capped PLGA with the low intrinsic viscosity is (70-90):(10-30), more preferably, 80:20.). In some embodiments, the weight ratio of the capped PLGA with the high intrinsic viscosity to the capped PLGA with the low intrinsic viscosity is (90:10)-(70:30), more preferably, 80:20. In some embodiments, the molar ratio of lactide to glycolide in the capped PLGA with the high intrinsic viscosity is within a range from 65:35 to 90:10, preferably, 75:25; and a molar ratio of lactide to glycolide in the capped PLGA with the low intrinsic viscosity is within a range from 50:50 to 75:25, preferably, 50:50. In some embodiments, the molar ratio of lactide to glycolide in the capped PLGA with the high intrinsic viscosity is within a range from 10:90 to 90:10, preferably, 50:50; and a molar ratio of lactide to glycolide in the capped PLGA with the low intrinsic viscosity is within a range from 50:50 to 75:25, preferably, 50:50.

In some embodiments, the two copolymers, i.e., the two PLGAs, are an uncapped PLGA with a high intrinsic viscosity of 0.4-0.9 dl/g, preferably, 0.45-0.8 dl/g, more preferably, 0.45-0.55 dl/g, and an uncapped PLGA with a low intrinsic viscosity of 0.1-0.35 dl/g, preferably, 0.1-0.3 dl/g, more preferably, 0.2-0.3 dl/g. A weight ratio of the uncapped PLGA with the high intrinsic viscosity to the uncapped PLGA with the low intrinsic viscosity is (50-95):(5-50), preferably, (70-90):(10-30), more preferably, 80:20. A molar ratio of lactide to glycolide in the uncapped PLGA with the high intrinsic viscosity is within a range from 65:35 to 90:10, preferably, 75:25; and a molar ratio of lactide to glycolide in the uncapped PLGA with the low intrinsic viscosity is within a range from 50:50 to 75:25, preferably, 50:50.

The intrinsic viscosity of PLGA is determined by preparing an about 0.5% (w/v) solution of PLGA in chloroform and determining the intrinsic viscosity of PLGA at 30° C. using a Cannon-Fenske glass capillary viscometer.

The PLGAs may also be a high molecular weight PLGA with a molecular weight of 20,000-145,000, preferably, 30,000-100,000, more preferably, 35,000-60,000 and a low molecular weight PLGA with a molecular weight of 4,000 to 45,000, preferably, 4,000-35,000, more preferably, 15,000-35,000. A weight ratio of the high molecular weight PLGA to the low molecular weight PLGA is (50-100):(0-50), preferably, (70-90):(10-30), more preferably, 80:20. A molar ratio of lactide to glycolide in the high molecular weight PLGA is within a range from 40:60 to 90:10, preferably, 50:50; and a molar ratio of lactide to glycolide in the low molecular weight PLGA is within a range from 50:50 to 75:25, preferably, 50:50. As used herein, the term “molecular weight” refers to “weight average molecular weight”, abbreviated as “molecular weight.”

For convenient description, hereinafter, the molar ratio of lactide to glycolide in PLGA and the intrinsic viscosity of PLGA are expressed in a bracket after PLGA. For example, “PLGA (50/50, 0.5 E)” refers to a lactide-glycolide copolymer having an intrinsic viscosity of 0.5 dl/g and a carboxyl terminal group, in which a molar ratio of lactide to glycolide is 50:50.

In some embodiments, the weight ratio of the capped PLGA (50/50, 0.5 E) with the high intrinsic viscosity to the uncapped PLGA (50/50, 0.25 A) with the low intrinsic viscosity in the present invention is 50:50.

Specifically, in some embodiments of the microsphere compositions of the present invention, the weight content of VLPs is between 0.01-20% and the weight content of PLGA is 80-99.09%, the weight ratio of the two PLGAs is 80:20, the molecular weight of the two PLGAs are 55,000˜85,000 and 15,000˜35,000, the intrinsic viscosity of the two PLGAs are 0.45˜0.55 dL/g and 0.2˜0.3 dL/g, and molar ratio of lactide to glycolide in the two PLGAs are 75:25 and 50:50, respectively.

In some embodiments, the composition includes one capped PLGA, ester capped, having a 50:50 ratio and an intrinsic viscosity of 0.45-0.6 dL/g.

In some embodiments, the 9vHPV vaccine/AAHS/PLGA microparticles, blank AAHS/PLGA microparticles were first made by a conventional water-in-oil-in-water (w/o/w) double emulsion process, isolated, and sterilized by Gamma irradiation. Subsequently, sterile HPV VLPs were rehydrated and loaded aseptically in blank microparticles at elevated temperature which accelerated mass transport. Each HPV serotype was loaded separately in individual containers and could be combined at desired ratio to prepare sterile 9V HPV/AAHS/PLGA microparticles.

To prepare sterile 9-valent HPV loaded PLGA microparticles, sterile HPV VLPs were added aseptically to blank microparticles and incubated at ambient temperature for 2 days followed by 37 C for another 2 days which accelerated mass transport. After loading, the particles were centrifuged and rinsed to remove unencapsulated HPV. Each HPV serotype was loaded separately in individual containers and could be combined at desired ratio to prepare sterile 9V HPV/AAHS/PLGA microparticles.

### Microspheres

The present disclosure is also directed to a method of preparing a core-shell particle wherein the core for microspheres could be dried in place or started as a solid intermediate. (See FIG. 1A.)

In some embodiments, a burst release formulation is provided. In some embodiments, the initial burst is provided by active ingredients present in a liquid formulation and a second burst is provided by the microparticles. (See FIG. 1B.) In some embodiments, a composition is provided that includes an HPV vaccine and PLGA. In some embodiments, the composition further includes AAHS. In some embodiments, the composition includes a core of PLGA that includes the HPV vaccine and a shell associated with the core, wherein the shell comprises PLGA.

Microspheres with a core-shell architecture can be particularly useful for controlled release applications. These types of microspheres can be produced using a variety of methods including atomization techniques using multi-phase nozzles, microfluidics devices to create double emulsions, coating processes in which a core is coated with coating solutions using atomized liquids or vapor deposition, or layered precipitation processes. Microspheres with a core-shell architecture can be designed such that each microsphere contains a single core or such that each microsphere contains more than one core. Core-shell microspheres can be particularly helpful with the encapsulation of materials that are sensitive to solvents or the local environment associated with materials comprising the shell. See FIG. 2A.

In some embodiments, a pulsatile drug delivery system is provided that includes a microneedle assembly including a plurality of microneedles filled with a therapeutic agent, the microneedles comprising biodegradable polymers, the microneedle assembly configured to release the therapeutic agent at predetermined times with a predetermined amount of the therapeutic agent while the microneedle assembly remains embedded in a patient.

### Spray Dried HPV VLPs

U.S. Pat. No. 10,772,947 describes how HPV VLPs bound to aluminum hydroxyphosphate sulfate (AAHS) can be lyophilized to maintain stability of the HPV VLPs as well as the AAHS particle size. Although this process is acceptable for drug product in vials, lyophilized powder does not provide the physical properties required for powder filling or direct compaction. Lyophilization is a sublimative process requiring freezing of the formulation and heating at temperatures below the collapse temperature of the product with subsequent secondary drying above collapse temperature resulting in a low-density powder. This could be translated into a bulk process, however, there are additional process steps to bulk dry, crush, and transfer before use resulting in significant process losses. In addition, there would be an extra blending step required to make particles that are free flowing for powder filling or compaction.

Spray drying is a bulk evaporative process which requires aspiration of solutions or suspensions and exposure to high temperatures to dry. The resulting powders have higher density than lyophilized powders and can be designed to be free-flowing powders with appropriate particle size for powder filling or compaction processes. However, the temperature used in the spray drying process require temperatures well above the HPV VLP melt temperature range of 59-61° C. In addition, evaporative drying of the aspirated droplets results in concentrating of AAHS as the droplet dries and this concentration is well documented to cause aggregation. The process described in this filing contains the process parameters and formulation components that produced a free-flowing powder of HPV VLPs bound to AAHS which stabilized the VLPs for potency and the prevented aggregation of the AAHS.

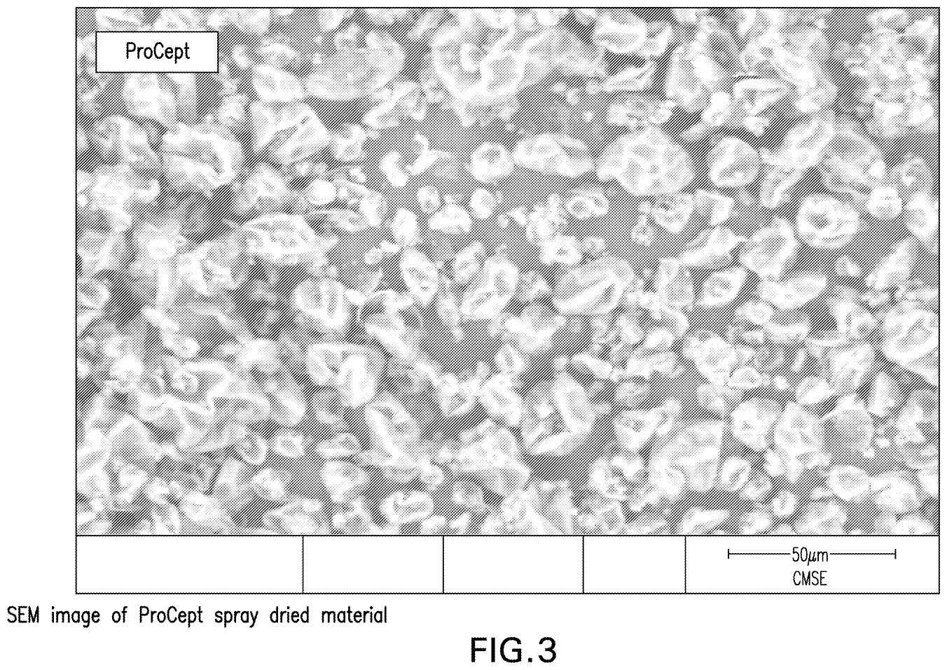

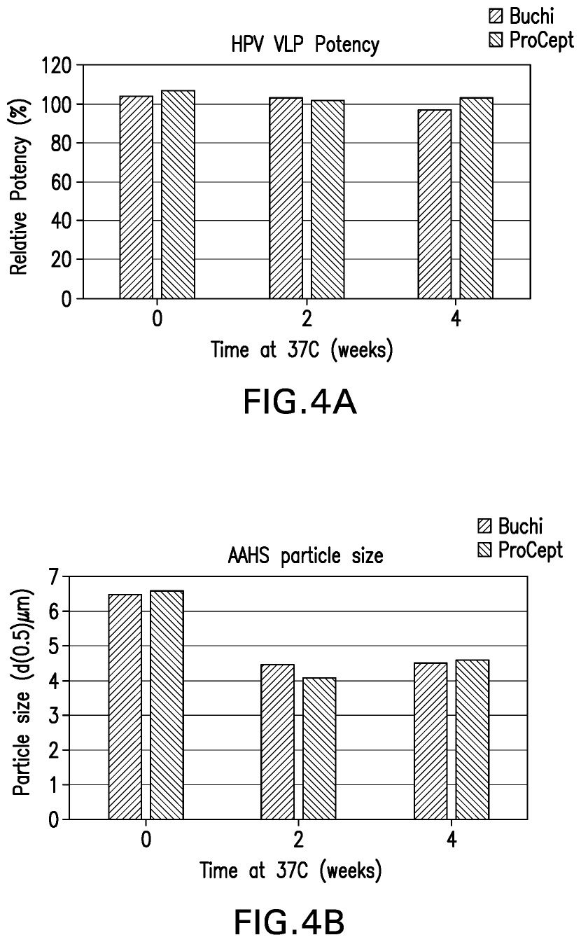

In some embodiments, stable spray dried HPV VLPs with AAHS are provided that are powder filled into vials/syringes for vaccine drug product or used as an intermediate for spray dried and compacted vaccine tablets. See FIGS. 3, 4A, and 4B. The process includes spray drying using a 2-3 fluid nozzle of the HPV VLPs bound to AAHS in the presence of a disaccharide (either trehalose or sucrose) with or without a tripeptide with a moisture content <6%.

### Vaccine Minitabs

The present disclosure is also directed to anew way of delivering HPV, bound and unbound to AAHS described below as the controlled release minitab. This invention describes a process for preparing a delayed release vaccine minitab that can be administered alone or in combination with an immediate release vaccine to provide protection against bacterial and viral pathogens. The liquid injection provides the initial immunization and the implant provides a delayed release at the injection site after weeks to months in the body and acts as the boost. This solves the challenge of individuals needing to schedule the follow-up with the healthcare provider. Specifically, a process of preparing a delayed release-vaccine minitab comprising of prophylactic agent was developed comprising of a.) providing a dry vaccine formulation containing ≤6% Moisture b.) compacting the dry formulation to obtain minitabs c.) coating the minitabs with a polymer coating composition; and d.) isolating said delayed release-vaccine minitab.

In some embodiments, a process for preparing a vaccine minitab is provided. In some embodiments, the vaccine minitab is administered to provide a controlled release of the vaccine to the intended recipient. In some embodiments, the vaccine minitab provides a controlled release vaccine that can be administered alone or in combination with an immediate release vaccine to provide protection against bacterial and viral pathogens. This challenge in obtaining broader coverage can be solved in part by administering the vaccine minitab at the same time as a vaccine liquid injection. The liquid injection provides the initial immunization and the vaccine minitab provides a controlled release at the injection site after weeks to months in the body and acts as the boost. This solves the challenge of individuals needing to schedule the follow-up with the healthcare provider.

In some embodiments, the minitablets can incorporate vaccines selected from subunit, conjugated and/or live virus (LVV) in adjuvanted and unadjuvanted form (i.e. Virus-like particles (VLPs) and aluminum hydroxyphosphate sulfate (AAHS)) in a range of dimensions and coated with a variety of polymers for either injection or surgical implantation to delay or prolong the release of the antigen. Compaction of materials into tablets require pressures in the range of 50 MPa to 400 MPa. These stresses occurring during compaction can damage proteins and due to the large size of many vaccines they are more vulnerable to these types of stresses than smaller proteins or small molecule pharmaceuticals. The formulation and binder combination are important to allow for adequate compressibility and compactability. Once compacted, the minitablet is coated by dropping the minitabs in a compatible anti-solvent containing the polymer of choice.

Minitabs of the invention include human papillomavirus VLPs. The core tablet may be either, a multiparticulate core: containing one or more pellet types, designed to release drug in the post-stomach portions of the GI tract, wherein the pellet types provide about 1 to 95% of total core tablet weight; or a mini-tab core: containing one or more mini-tabs also designed to release drug in the post-stomach GI tract, wherein the min-tabs provide about 1 to 95% of total core tablet weight. The enteric polymer layer can be produced by spraying a number of different types of enteric polymers on top of the above-described core tablet. Thus, the enteric polymer coating delays the disintegration of the core tablet until the core tablet reaches the intestine. Additionally, the multiparticulates or mini-tabs comprising the core tablet may be formulated with further delayed and/or modified release materials, to thereby further delay and/or modify release of the active drug agents released therefrom.

In one embodiment the immediate release portion is an immediate release drug layer that can be sprayed on top of the enteric-coated core tablet to thereby serve as an immediate releasing portion of the drug delivery system. In one embodiment of the invention the immediate release drug layer completely engulfs the enteric polymer coated core tablet. In one embodiment of the invention the immediate release drug layer does not completely engulf the enteric polymer coating of the core tablet; in this embodiment a portion of the enteric polymer coating is exposed.

### Vaccine Implant

The present disclosure is also directed to a process of preparing a delayed release-vaccine implant comprising of prophylactic agent comprising of a method of a.) providing a dry vaccine formulation containing ≤5% Moisture b.) mixing the dry vaccine formulation with a molten polymer; c.) preparing the implant by extruding the polymer vaccine matrix; d.) coating the implant with a polymer coating composition; e.) isolating said delayed release-vaccine implant wherein extrudate temperatures occurs above HPV VLP Tm without degrading HPV VLP.

In some embodiments, a process is provided for preparing a vaccine implant, which is suitable for subdermal implantation. See, e.g. FIG. 2B. In some embodiments, the vaccine implant is administered to provide a delayed release of the vaccine to the intended recipient. In some embodiments, the vaccine implant provides a controlled release vaccine that can be administered alone or in combination with an immediate release vaccine to provide protection against bacterial and viral pathogens. This challenge in obtaining broader coverage can be solved in part with this implant that can be administered at the same time as the liquid injection. The liquid injection provides the initial immunization and the implant provides a delayed release at the injection site after weeks to months in the body and acts as the boost. This solves the challenge of individuals needing to schedule the follow-up with the healthcare provider.

### VLPs

As stated above, the pharmaceutical compositions and formulations of the present invention comprise at least one HPV VLP type, such as HPV 16 or 18. In particular embodiments of the compositions disclosed herein, the vaccine further comprises VLPs of at least one additional HPV type. In further embodiments, the at least one additional HPV type is selected from the group consisting of 6, 11, 16, 18, 26, 31, 33, 35, 39, 45, 51, 52, 53, 55, 56, 58, 59, 66, 68, 69, 70, 73, and 82. In some embodiments, the at least one additional HPV type includes HPV 16 and 18. In some embodiments, the at least one additional HPV type includes HPV 6, 11, 16, and 18. In some embodiments, the at least one additional HPV type includes HPV 6, 18, 52, and 58. In some embodiments, the at least one additional HPV type includes HPV 6, 11, 16, 18, 31, 45, 52, and 58. In some embodiments, the at least one additional HPV type includes HPV 6, 11, 16, 18, 33, 45, 52, and 58. In some embodiments, the at least one additional HPV type includes HPV 6, 11, 16, 18, 31, 33, 45, 52, and 58. In some embodiments, the at least one additional HPV type includes 6, 11, 16, 18, 31, 33, 45, 52, and 59. In some embodiments, the at least one additional HPV type includes HPV 6, 11, 16, 18, 31, 33, 45, 53, and 58. In some embodiments, the at least one additional HPV type includes HPV 6, 11, 16, 18, 31, 33, 45, 53, and 59. In some embodiments, the at least one additional HPV type includes HPV 6, 11, 16, 18, 31, 33, 35, 45, 52, and 58. In some embodiments, the at least one additional HPV type includes HPV 6, 11, 16, 18, 31, 33, 35, 45, 52, 58, and 59. In some embodiments, the at least one additional HPV type includes HPV 6, 11, 16, 18, 31, 33, 45, 52, 58, 59, and 68. In some embodiments, the at least one additional HPV type includes HPV 6, 11, 16, 18, 31, 33, 35, 39, 45, 51, 52, 56, 58, and 59. In some embodiments, the at least one additional HPV type includes HPV 6, 11, 16, 18, 26, 31, 33, 35, 45, 51, 52, 58, 59, and 69. In some embodiments, the at least one additional HPV type includes HPV 6, 11, 16, 18, 26, 31, 33, 35, 39, 45, 51, 52, 58, 59, 68, 69, and 70. In some embodiments, the at least one additional HPV type includes HPV 6, 11, 16, 18, 26, 31, 33, 35, 39, 45, 51, 52, 53, 56, 58, 59, 66, 68, 69, and 70. The pharmaceutical compositions of the present invention comprise HPV VLPs comprised of recombinant L1 or recombinant L1+L2 proteins of HPV. HPV L1 or L1+L2 protein can be expressed recombinantly by molecular cloning of L1 or L1+L2 DNA into an expression vector containing a suitable promoter and other appropriate transcription regulatory elements, and transferred into prokaryotic or eukaryotic host cells to produce recombinant protein. Techniques for such manipulations are fully described by Sambrook et al. (Molecular Cloning: A Laboratory Manual; Cold Spring Harbor Laboratory, Cold Spring Harbor, New York, (1989)), which is hereby incorporated by reference. VLPs can self-assemble when L1 protein is recombinantly expressed in a host cell.

The recombinant HPV L1 proteins of the present invention may be any full-length L1 protein sequence that can be found in nature or any mutated or truncated L1 protein that is capable of self-assembling into VLPs. In particular embodiments of the invention, the pharmaceutical compositions and vaccines described herein comprise HPV VLPs comprised of recombinant HPV L1 protein and do not contain HPV L2 protein. In certain embodiments, the vaccine compositions or pharmaceutical compositions described herein comprise HPV VLPs comprised of a full-length recombinant HPV L1 protein. In other embodiments, the HPV VLPs are comprised of truncated HPV L1 protein, e.g. L1 protein that are truncated at the C-terminal end. L1 protein sequences for use in the present invention can be determined by isolating DNA from one or more clinical samples containing an HPV type of choice, determining the sequence of the HPV L1 DNA sequence, and translating the DNA sequence into an amino acid sequence using the genetic code. Many exemplary L1 sequences suitable for use in the present invention can be found in the literature. See, e.g., U.S. Pat. Nos. 5,820,870; 7,250,170; 7,276,243; 7,482,428; 7,976,848; 7,498,036; 7,700,103; 7,744,892; and U.S. Pat. No. 5,437,951; Kirii et al. (Virology 185(1): 424-427 (1991)). Further L1 proteins that are useful in the compositions and formulations of the present invention include biologically active fragments and/or mutants of an HPV L1 sequence, including but not necessarily limited to amino acid substitutions, deletions, additions, amino terminal truncations and carboxy-terminal truncations, such that these mutations provide for L1 proteins or protein fragments that are capable of forming a VLP. See, e.g., International Publication WO 2006/114312 and U.S. Pat. No. 6,599,508. Appropriate host cells for the expression of recombinant HPV L1 or recombinant L1+L2 and subsequent self-assembly of VLPs include, but are not limited to yeast cells, insect cells, mammalian cells or bacteria. In exemplary embodiments of the invention, the VLPs are produced in yeast cells such as a yeast selected from the group consisting of: Saccharomyces cerevisiae, Hansenula polvnorpha, Pichia pastoris, Kluyvermyces fragilis, Kluveromyces lactis, and Schizosaccharomyces pombe. In particular embodiments, the HPV VLPs are produced in Saccharomyces cerevisiae cells. Expression of HPV VLPs in yeast cells offers the advantages of being cost-effective and easily adapted to large-scale growth in fermenters.

The present invention also includes pharmaceutical compositions comprising mutant forms of HPV VLPs, such as HPV VLPs that comprise biologically active fragments and/or mutants of an HPV L1 or L2 protein, including but not necessarily limited to amino acid substitutions, deletions, additions, amino terminal truncations and carboxy-terminal truncations such that these mutations provide for proteins or protein fragments of therapeutic or prophylactic use and would be useful for HPV VLP vaccine development. Any such mutant form of an HPV L1 protein should be capable of forming VLPs and of provoking an immune response against the desired HPV type when administered to a human.

Additionally, one of skill in the art will recognize that the HPV L1 or L1+L2 proteins, which are used to self-assemble VLPs for inclusion in the compositions disclosed herein, may be encoded by a full-length wild-type HPV L1 or L2 polynucleotide, or may be encoded by a fragment or mutant of the known wild-type sequence. Wild-type polynucleotide sequences that encode mRNA expressing HPV L1 or L2 protein are available in the art. Any mutant polynucleotide will encode either a protein or protein fragment which at least substantially mimics the pharmacological properties of an HPV L1 or L2 protein, including the ability to form VLPs that are able to provoke an immune response against the HPV type of interest when administered to a human. Any such polynucleotide includes but is not necessarily limited to: nucleotide substitutions, deletions, additions, amino-terminal truncations and carboxy-terminal truncations.

The amount of virus-like particles of each HPV type to be included in the formulations and compositions of the present invention will depend on the immunogenicity of the expressed gene product. In general, a therapeutically effective dose of VLPs of any of the at least one HPV type is about 1 μg to about 300 μg. In some embodiments, a therapeutically effective dose of VLPs of any of the at least one HPV type is about 1 μg to 200 μg. In some embodiments, a therapeutically effective dose of VLPs of any of the at least one HPV type is about 1 μg to 100 μg. In some embodiments, a therapeutically effective dose of VLPs of any of the at least one HPV type is about 10 μg to 200 μg. In some embodiments, a therapeutically effective dose of VLPs of any of the at least one HPV type is about 10 μg to 100 μg. In some embodiments, a therapeutically effective dose of VLPs of any of the at least one HPV type is about 10 μg to 80 μg. In some embodiments, a therapeutically effective dose of VLPs of any of the at least one HPV type is about preferably about 20 μg to 60 μg.

In some embodiments, a 0.5 mL dose of a composition or vaccine including VLPs of the at least one HPV type includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein,
10-120 μg of VLPs of HPV Type 31 L1 protein,
10-120 μg of VLPs of HPV Type 33 L1 protein,
10-120 μg of VLPs of HPV Type 45 L1 protein,
10-120 μg of VLPs of HPV Type 52 L1 protein, and
10-120 μg of VLPs of HPV Type 58 L1 protein.

In some embodiments, a 0.5 mL dose of a composition or vaccine including VLPs of the at least one HPV type includes:
15-120 μg of VLPs of HPV Type 6 L1 protein,
20-150 μg of VLPs of HPV Type 11 L1 protein,
30-210 μg of VLPs of HPV Type 16 L1 protein,
20-150 μg of VLPs of HPV Type 18 L1 protein,
10-90 μg of VLPs of HPV Type 31 L1 protein,
10-90 μg of VLPs of HPV Type 33 L1 protein,
10-90 μg of VLPs of HPV Type 45 L1 protein,
10-90 μg of VLPs of HPV Type 52 L1 protein, and
10-90 μg of VLPs of HPV Type 58 L1 protein.

In some embodiments, a 0.5 mL dose of a composition or vaccine including VLPs of the at least one HPV type includes:
15-80 μg of VLPs of HPV Type 6 L1 protein,
20-100 μg of VLPs of HPV Type 11 L1 protein,
30-140 μg of VLPs of HPV Type 16 L1 protein,
20-100 μg of VLPs of HPV Type 18 L1 protein,
10-60 μg of VLPs of HPV Type 31 L1 protein,
10-60 μg of VLPs of HPV Type 33 L1 protein,
10-60 μg of VLPs of HPV Type 45 L1 protein,
10-60 μg of VLPs of HPV Type 52 L1 protein, and
10-60 μg of VLPs of HPV Type 58 L1 protein.

In some embodiments, a 0.5 mL dose of a composition or vaccine including VLPs of the at least one HPV type includes:
15-40 μg of VLPs of HPV Type 6 L1 protein,
20-50 μg of VLPs of HPV Type 11 L1 protein,
30-70 μg of VLPs of HPV Type 16 L1 protein,
20-50 μg of VLPs of HPV Type 18 L1 protein,
10-30 μg of VLPs of HPV Type 31 L1 protein,
10-30 μg of VLPs of HPV Type 33 L1 protein,
10-30 μg of VLPs of HPV Type 45 L1 protein,
10-30 μg of VLPs of HPV Type 52 L1 protein, and
10-30 μg of VLPs of HPV Type 58 L1 protein.

In some embodiments, a 0.5 mL dose of a composition or vaccine including VLPs of the at least one HPV type includes:
90 μg of VLPs of HPV Type 6 L1 protein,
120 μg of VLPs of HPV Type 11 L1 protein,
180 μg of VLPs of HPV Type 16 L1 protein,
120 μg of VLPs of HPV Type 18 L1 protein,
60 μg of VLPs of HPV Type 31 L1 protein,
60 μg of VLPs of HPV Type 33 L1 protein,
60 μg of VLPs of HPV Type 45 L1 protein,
60 μg of VLPs of HPV Type 52 L1 protein, and
60 μg of VLPs of HPV Type 58 L1 protein.

In some embodiments, a 0.5 mL dose of a composition or vaccine including VLPs of the at least one HPV type includes:
60 μg of VLPs of HPV Type 6 L1 protein,
80 μg of VLPs of HPV Type 11 L1 protein,
120 μg of VLPs of HPV Type 16 L1 protein,
80 μg of VLPs of HPV Type 18 L1 protein,
40 μg of VLPs of HPV Type 31 L1 protein,
40 μg of VLPs of HPV Type 33 L1 protein,
40 μg of VLPs of HPV Type 45 L1 protein,
40 μg of VLPs of HPV Type 52 L1 protein, and
40 μg of VLPs of HPV Type 58 L1 protein.

In some embodiments, a 0.5 mL dose of a composition or vaccine including VLPs of the at least one HPV type includes:
30 μg of VLPs of HPV Type 6 L1 protein,
40 μg of VLPs of HPV Type 11 L1 protein,
60 μg of VLPs of HPV Type 16 L1 protein,
40 μg of VLPs of HPV Type 18 L1 protein,
20 μg of VLPs of HPV Type 31 L1 protein,
20 μg of VLPs of HPV Type 33 L1 protein,
20 μg of VLPs of HPV Type 45 L1 protein,
20 μg of VLPs of HPV Type 52 L1 protein, and
20 μg of VLPs of HPV Type 58 L1 protein.

### Aluminum Adjuvants

The aluminum adjuvant of the present invention may be in the form of aluminum hydroxide (Al(OH)₃), aluminum phosphate (AlPO₄), aluminum hydroxyphosphate, amorphous aluminum hydroxyphosphate sulfate (AAHS) or so-called “alum” (KAl(SO₄)— 12H₂O) (see Klein et al, Analysis of aluminum hydroxyphosphate vaccine adjuvants by (27)A1 MAS NMR., J Pharm. Sci. 89(3): 311-21 (2000)). In exemplary embodiments of the invention provided herein, the aluminum adjuvant is aluminum hydroxyphosphate or AAHS. The ratio of phosphate to aluminum in the aluminum adjuvant can range from 0 to 1.3. In embodiments of this aspect of the invention, the phosphate to aluminum ratio is within the range of 0.1 to 0.70. In some embodiments, the phosphate to aluminum ratio is within the range of 0.2 to 0.50.

One of skill in the art will be able to determine an optimal dosage of aluminum adjuvant that is both safe and effective at increasing the immune response to the targeted HPV type(s). For a discussion of the safety profile of aluminum, as well as amounts of aluminum included in FDA-licensed vaccines, see Baylor et al., Vaccine 20: S18-S23 (2002). In some embodiments, the aluminum adjuvant is present in the amount of about 100 to 3600 μg/dose (200 to 7200 μg/mL concentration). In some embodiments, the aluminum adjuvant is present in the amount of about 100 to 2700 μg/dose (200 to 5400 μg/mL concentration). In some embodiments, the aluminum adjuvant is present in the amount of about 100 to 1800 μg/dose (200 to 3600 μg/mL concentration). In some embodiments, the aluminum adjuvant is present in the amount of about 100 to 900 μg/dose (200 to 1800 μg/mL concentration). In some embodiments of the formulations and compositions of the present invention, there is between 200 and 300 μg aluminum adjuvant per dose of vaccine. In alternative embodiments of the formulations and compositions of the present invention, there is between 300 and 500 μg aluminum adjuvant per dose of vaccine. In alternative embodiments of the formulations and compositions of the present invention, there is between 400 and 1200 μg aluminum adjuvant per dose of vaccine. In alternative embodiments of the formulations and compositions of the present invention, there is between 1200 and 2000 μg aluminum adjuvant per dose of vaccine. In some embodiments of the formulations and compositions of the present invention, there is less than 2000 μg aluminum adjuvant per dose of vaccine. In some embodiments of the formulations and compositions of the present invention, there is less than 1500 μg aluminum adjuvant per dose of vaccine. In some embodiments of the formulations and compositions of the present invention, there is less than 1000 μg aluminum adjuvant per dose of vaccine. In some embodiments of the formulations and compositions of the present invention, there is less than 500 μg aluminum adjuvant per dose of vaccine. In some embodiments of the formulations and compositions of the present invention, there is less than 400 μg aluminum adjuvant per dose of vaccine. In some embodiments of the formulations and compositions of the present invention, there is less than 300 μg aluminum adjuvant per dose of vaccine. In some embodiments of the formulations and compositions of the present invention, there is less than 200 μg aluminum adjuvant per dose of vaccine. In some embodiments of the formulations and compositions of the present invention, there is less than 100 μg aluminum adjuvant per dose of vaccine.

### Chitosan Adjuvants

Chitin, a polymer of N-acetyglucosamine (i.e., (1-4)-2-acetamido-2-deoxypβ-d-glucan), is a significant component of the body of all crustaceans and is also present in the exoskeleton and the cell wall of fungi, insects, and yeast. Chitosan, i.e., α-(1-4)-2-amino-2-deoxy-β-d-glucan, is the mostly deacetylated form of the naturally occurring polysaccharide chitin. Chitosan is typically formed by deacetylation of chitin in the presence of alkali. The term “chitosan” refers to the class of molecules having a degree of deacetylation that is different from chitin. For example, molecules having a deacetylation below 75% is consider chitin. In contrast, molecules having a deacetylation above 75% is considered chitosan. The deacetylation of chitosan powder may be measured via NMR, UV (EP method), or IR. The deacetylation of chitosan in solution may be measured via CZE (capillary zone electrophoresis), GC-MS, ion chromatography, and SEC-UV.

In some embodiments, the chitosan adjuvant includes a chitosan. In some embodiments, the chitosan adjuvant includes a chitosan having a deacetylation of 75% or greater. In some embodiments, the chitosan adjuvant includes chitosan that has a degree of deacetylation in the range of about 75-99%. In some embodiments, the chitosan adjuvant includes chitosan that has a degree of deacetylation in the range of about 85-99%. In some embodiments, the chitosan adjuvant includes chitosan that has a degree of deacetylation greater than 90%. In some embodiments, the chitosan adjuvant includes chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. Deacetylation may be calculated according to any of the methods described herein.

In some embodiments, the chitosan adjuvant includes chitosan that is a water-soluble chitosan having a degree of deacetylation in the range of about 75-99%. In some embodiments, the chitosan adjuvant includes water-soluble chitosan that has a degree of deacetylation in the range of about 75-99%. In some embodiments, the chitosan adjuvant includes water-soluble chitosan that has a degree of deacetylation in the range of about 85-99%. In some embodiments, the chitosan adjuvant includes water-soluble chitosan that has a degree of deacetylation greater than 90%. In some embodiments, the chitosan adjuvant includes a water-soluble chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. In some embodiments, the water-soluble chitosan is chitosan hydrochloride. Deacetylation may be calculated according to any of the methods described herein.

In some embodiments, the chitosan adjuvant includes an acid-soluble chitosan that has a degree of deacetylation in the range of about 75-99%. In some embodiments, the acid-soluble chitosan is chitosan hydrochloride. In some embodiments, the chitosan adjuvant includes an acid-soluble chitosan that has a degree of deacetylation in the range of about 85-99%. In some embodiments, the chitosan adjuvant includes an acid-soluble chitosan that has a degree of deacetylation greater than 90%. In some embodiments, the chitosan adjuvant includes an acid-soluble chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. Deacetylation may be calculated according to any of the methods described above.

In some embodiments, the chitosan adjuvant includes a chitosan having a viscosity in the range of about 1 cP to about 200 cP when measured with a viscosimeter (e.g. Brookfield DVII+pro) at 20° C. at a standard concentration (e.g. 1% in 1% acetic acid). In some embodiments, the chitosan has viscosity in the range of about 1 cP to about 100 cP. In some embodiments, the chitosan has viscosity in the range of about 5 cP to about 100 cP. In some embodiments, the chitosan has viscosity in the range of about 1 cP to about 50 cP. In some embodiments, the chitosan has viscosity in the range of about 5 cP to about 50 cP. In some embodiments, the chitosan has viscosity in the range of about 1 cP to about 25 cP. In some embodiments, the chitosan has viscosity in the range of about 5 cP to about 25 cP. In some embodiments, the chitosan has viscosity in the range of about 1 cP to about 20 cP. In some embodiments, the chitosan has viscosity in the range of about 5 cP to about 20 cP. In some embodiments, the chitosan has viscosity in the range of about 1 cP to about 15 cP. In some embodiments, the chitosan has viscosity in the range of about 5 cP to about 15 cP. In some embodiments, the chitosan has viscosity in the range of about 1 cP to about 10 cP. In some embodiments, the chitosan has viscosity in the range of about 5 cP to about 10 cP. In some embodiments, the chitosan has a viscosity less than 100. In some embodiments, the chitosan has a viscosity less than 75. In some embodiments, the chitosan has a viscosity less than 50. In some embodiments, the chitosan has a viscosity less than 40. In some embodiments, the chitosan has a viscosity less than 30. In some embodiments, the chitosan has a viscosity less than 20. In some embodiments, the chitosan has a viscosity less than 15. In some embodiments, the chitosan has a viscosity less than 10. In some embodiments, the chitosan has a viscosity less than 5. In some embodiments, the chitosan has viscosity of 1 cP, 2 cP, 3 cP, 4 cP, 5 cp, 6 cp, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, or 50 cP.

In some embodiments, the chitosan adjuvant includes a water-soluble chitosan having a viscosity in the range of about 1 cP to about 200 cP. In some embodiments, the water-soluble chitosan includes chitosan hydrochloride having a viscosity in the range of about 1 cP to about 200 cP when measured with a viscosimeter (e.g. Brookfield DVII+pro) at 20° C. at a standard concentration (e.g. 1% in 1% acetic acid. In some embodiments, the chitosan hydrochloride has viscosity in the range of about 1 cP to about 100 cP. In some embodiments, the chitosan hydrochloride has viscosity in the range of about 5 cP to about 100 cP. In some embodiments, the chitosan hydrochloride has viscosity in the range of about 1 cP to about 50 cP. In some embodiments, the chitosan hydrochloride has viscosity in the range of about 5 cP to about 50 cP. In some embodiments, the chitosan hydrochloride has viscosity in the range of about 1 cP to about 25 cP. In some embodiments, the chitosan hydrochloride has viscosity in the range of about 5 cP to about 25 cP. In some embodiments, the chitosan hydrochloride has viscosity in the range of about 1 cP to about 20 cP. In some embodiments, the chitosan hydrochloride has viscosity in the range of about 5 cP to about 20 cP. In some embodiments, the chitosan hydrochloride has viscosity in the range of about 1 cP to about 15 cP. In some embodiments, the chitosan hydrochloride has viscosity in the range of about 5 cP to about 15 cP. In some embodiments, the chitosan hydrochloride has viscosity in the range of about 1 cP to about 10 cP. In some embodiments, the chitosan hydrochloride has viscosity in the range of about 5 cP to about 10 cP. In some embodiments, the chitosan hydrochloride has a viscosity less than 100. In some embodiments, the chitosan hydrochloride has a viscosity less than 75. In some embodiments, the chitosan hydrochloride has a viscosity less than 50. In some embodiments, the chitosan hydrochloride has a viscosity less than 40. In some embodiments, the chitosan hydrochloride has a viscosity less than 30. In some embodiments, the chitosan hdyrochloride has a viscosity less than 20. In some embodiments, the chitosan hydrochloride has a viscosity less than 15. In some embodiments, the chitosan hydrochloride has a viscosity less than 10. In some embodiments, the chitosan hydrochloride has a viscosity less than 5. In some embodiments, the chitosan hydrochloride has viscosity of 1 cP, 2 cP, 3 cP, 4 cP, 5 cp, 6 cp, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, or 50 cP.

In some embodiments, the chitosan adjuvant includes an acid-soluble chitosan having a viscosity in the range of about 1 cP to about 200 cP when measured with a viscosimeter (e.g. Brookfield DVII+pro) at 20° C. at a standard concentration (e.g. 1% in 1% acetic acid). In some embodiments, the acid-soluble chitosan has viscosity in the range of about 1 cP to about 100 cP. In some embodiments, the acid-soluble chitosan has viscosity in the range of about 5 cP to about 100 cP. In some embodiments, the acid-soluble chitosan has viscosity in the range of about 1 cP to about 50 cP. In some embodiments, the acid-soluble chitosan has viscosity in the range of about 5 cP to about 50 cP. In some embodiments, the acid-soluble chitosan has viscosity in the range of about 1 cP to about 25 cP. In some embodiments, the acid-soluble chitosan has viscosity in the range of about 5 cP to about 25 cP. In some embodiments, the acid-soluble chitosan has viscosity in the range of about 1 cP to about 20 cP. In some embodiments, the acid-soluble chitosan has viscosity in the range of about 5 cP to about 20 cP. In some embodiments, the acid-soluble chitosan has viscosity in the range of about 1 cP to about 15 cP. In some embodiments, the acid-soluble chitosan has viscosity in the range of about 5 cP to about 15 cP. In some embodiments, the acid-soluble chitosan has viscosity in the range of about 1 cP to about 10 cP. In some embodiments, the acid-soluble chitosan has viscosity in the range of about 5 cP to about 10 cP. In some embodiments, the acid-soluble chitosan has a viscosity less than 100. In some embodiments, the acid-soluble chitosan has a viscosity less than 75. In some embodiments, the acid-soluble chitosan has a viscosity less than 50. In some embodiments, the acid-soluble chitosan has a viscosity less than 40. In some embodiments, the acid-soluble chitosan has a viscosity less than 30. In some embodiments, the acid-soluble chitosan has a viscosity less than 20. In some embodiments, the acid-soluble chitosan has a viscosity less than 15. In some embodiments, the acid-soluble chitosan has a viscosity less than 10. In some embodiments, the acid-soluble chitosan has a viscosity less than 5. In some embodiments, the acid-soluble chitosan has viscosity of 1 cP, 2 cP, 3 cP, 4 cP, 5 cp, 6 cp, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, or 50 cP.

In some embodiments, the chitosan adjuvant includes a water-soluble chitosan having a degree of deacetylation greater than 75% and a viscosity of less than 50 cP. In some embodiments, the water-soluble chitosan includes chitosan hydrochloride having a degree of deacetylation greater than 75% and a viscosity of less than 50 cP. In some embodiments, the chitosan adjuvant includes chitosan hydrochloride having a degree of deacetylation greater than 75% and a viscosity of less than 40 cP. In some embodiments, the chitosan hydrochloride has a degree of deacetylation greater than 75% and a viscosity of less than 30 cP. In some embodiments, the chitosan hydrochloride has a degree of deacetylation greater than 75% and a viscosity of less than 20 cP. In some embodiments, the chitosan hydrochloride has a degree of deacetylation greater than 75% and a viscosity of less than 10 cP. In some embodiments, the chitosan hydrochloride has a degree of deacetylation greater than 75% and a viscosity of less than 5 cP. In some embodiments, the chitosan hydrochloride has a degree of deacetylation greater than 85% and a viscosity of less than 50 cP. In some embodiments, the chitosan hydrochloride has a degree of deacetylation greater than 85% and a viscosity of less than 40 cP. In some embodiments, the chitosan hydrochloride has a degree of deacetylation greater than 85% and a viscosity of less than 30 cP. In some embodiments, the chitosan hydrochloride has a degree of deacetylation greater than 85% and a viscosity of less than 20 cP. In some embodiments, the chitosan hydrochloride has a degree of deacetylation greater than 85% and a viscosity of less than 10 cP. In some embodiments, the chitosan hydrochloride has a degree of deacetylation greater than 85% and a viscosity of less than 5 cP. In some embodiments, the chitosan hydrochloride has a degree of deacetylation greater than 90% and a viscosity of less than 50 cP. In some embodiments, the chitosan hydrochloride has a degree of deacetylation greater than 90% and a viscosity of less than 40 cP. In some embodiments, the chitosan hydrochloride has a degree of deacetylation greater than 90% and a viscosity of less than 30 cP. In some embodiments, the chitosan hydrochloride has a degree of deacetylation greater than 90% and a viscosity of less than 20 cP. In some embodiments, the chitosan hydrochloride has a degree of deacetylation greater than 90% and a viscosity of less than 10 cP. In some embodiments, the chitosan hydrochloride has a degree of deacetylation greater than 90% and a viscosity of less than 5 cP. In some embodiments, the chitosan hydrochloride has a degree of deacetylation greater than 95% and a viscosity of less than 50 cP. In some embodiments, the chitosan hydrochloride has a degree of deacetylation greater than 95% and a viscosity of less than 40 cP. In some embodiments, the chitosan hydrochloride has a degree of deacetylation greater than 95% and a viscosity of less than 30 cP. In some embodiments, the chitosan hydrochloride has a degree of deacetylation greater than 95% and a viscosity of less than 20 cP. In some embodiments, the chitosan hydrochloride has a degree of deacetylation greater than 95% and a viscosity of less than 10 cP. In some embodiments, the chitosan hydrochloride has a degree of deacetylation greater than 95% and a viscosity of less than 5 cP.

In some embodiments, the chitosan adjuvant includes an acid-soluble chitosan having a degree of deacetylation greater than 75% and a viscosity of less than 50 cP. In some embodiments, the acid-soluble chitosan has a degree of deacetylation greater than 75% and a viscosity of less than 40 cP. In some embodiments, the acid-soluble chitosan has a degree of deacetylation greater than 75% and a viscosity of less than 30 cP. In some embodiments, the acid-soluble chitosan has a degree of deacetylation greater than 75% and a viscosity of less than 20 cP. In some embodiments, the acid-soluble chitosan has a degree of deacetylation greater than 75% and a viscosity of less than 10 cP. In some embodiments, the acid-soluble chitosan has a degree of deacetylation greater than 75% and a viscosity of less than 5 cP. In some embodiments, the acid-soluble chitosan has a degree of deacetylation greater than 85% and a viscosity of less than 50 cP. In some embodiments, the acid-soluble chitosan has a degree of deacetylation greater than 85% and a viscosity of less than 40 cP. In some embodiments, the acid-soluble chitosan has a degree of deacetylation greater than 85% and a viscosity of less than 30 cP. In some embodiments, the acid-soluble chitosan has a degree of deacetylation greater than 85% and a viscosity of less than 20 cP. In some embodiments, the acid-soluble chitosan has a degree of deacetylation greater than 85% and a viscosity of less than 10 cP. In some embodiments, the acid-soluble chitosan has a degree of deacetylation greater than 85% and a viscosity of less than 5 cP. In some embodiments, the acid-soluble chitosan has a degree of deacetylation greater than 90% and a viscosity of less than 50 cP. In some embodiments, the acid-soluble chitosan has a degree of deacetylation greater than 90% and a viscosity of less than 40 cP. In some embodiments, the acid-soluble chitosan has a degree of deacetylation greater than 90% and a viscosity of less than 30 cP. In some embodiments, the acid-soluble chitosan has a degree of deacetylation greater than 90% and a viscosity of less than 20 cP. In some embodiments, the acid-soluble chitosan has a degree of deacetylation greater than 90% and a viscosity of less than 10 cP. In some embodiments, the acid-soluble chitosan has a degree of deacetylation greater than 90% and a viscosity of less than 5 cP. In some embodiments, the acid-soluble chitosan has a degree of deacetylation greater than 95% and a viscosity of less than 50 cP. In some embodiments, the acid-soluble chitosan has a degree of deacetylation greater than 95% and a viscosity of less than 40 cP. In some embodiments, the acid-soluble chitosan has a degree of deacetylation greater than 95% and a viscosity of less than 30 cP. In some embodiments, the acid-soluble chitosan has a degree of deacetylation greater than 95% and a viscosity of less than 20 cP. In some embodiments, the acid-soluble chitosan has a degree of deacetylation greater than 95% and a viscosity of less than 10 cP. In some embodiments, the acid-soluble chitosan has a degree of deacetylation greater than 95% and a viscosity of less than 5 cP.

In some embodiments, the chitosan adjuvant includes chitosan in the amount of about 0.1 μg to about 200 mg. In some embodiments, the chitosan adjuvant includes chitosan in the amount of about 0.1 μg to about 100 mg. In some embodiments, the chitosan adjuvant includes chitosan in the amount of about 0.1 μg to about 50 mg. In some embodiments, the chitosan adjuvant includes chitosan in the amount of about 0.1 μg to about 25 mg. In some embodiments, the chitosan adjuvant includes chitosan in the amount of about 0.1 μg to about 20 mg. In some embodiments, the chitosan adjuvant includes chitosan in the amount of about 0.1 μg to about 100 mg. In some embodiments, the chitosan adjuvant includes less than 100 mg of chitosan. In some embodiments, the chitosan adjuvant includes less than 90 mg of chitosan. In some embodiments, the chitosan adjuvant includes less than 80 mg of chitosan. In some embodiments, the chitosan adjuvant includes less than 70 mg of chitosan. In some embodiments, the chitosan adjuvant includes less than 60 mg of chitosan. In some embodiments, the chitosan adjuvant includes less than 50 mg of chitosan. In some embodiments, the chitosan adjuvant includes less than 40 mg of chitosan. In some embodiments, the chitosan adjuvant includes less than 30 mg of chitosan. In some embodiments, the chitosan adjuvant includes less than 20 mg of chitosan. In some embodiments, the chitosan adjuvant includes less than 10 mg of chitosan. In some embodiments, the chitosan adjuvant includes less than 5 mg of chitosan. In some embodiments, the chitosan is water-soluble chitosan. In some embodiments, the chitosan is acid-soluble chitosan. In some embodiments, the chitosan is chitosan hydrochloride.

In some embodiments, the chitosan adjuvant includes a buffer. In some embodiments, the buffer is selected from any pharmaceutically acceptable buffer, including acetic acid, histidine, citrate, Bis-Tris, HEPES, phosphate, MES, and combinations thereof. In some embodiments, the buffer is present in the amount of 1 mMol to about 100 mMol of the chitosan adjuvant.

In some embodiments, the chitosan adjuvant includes a water-soluble chitosan and a buffer. In some embodiments, the chitosan adjuvant includes chitosan hydrochloride and a buffer. In some embodiments, the chitosan adjuvant includes a buffer and a water-soluble chitosan having a deacetylation of greater than 75% and a viscosity of 1 cp to 200 cp. In some embodiments, the chitosan adjuvant includes a buffer and chitosan hydrochloride having a deacetylation of greater than 75% and a viscosity of 1 cp to 200 cp. In some embodiments, the chitosan adjuvant includes chitosan hydrochloride and a histidine buffer. In some embodiments, the chitosan adjuvant includes a buffer and chitosan hydrochloride having a deacetylation of greater than 75% and a viscosity of 1 cp to 200 cp. In some embodiments, the chitosan adjuvant includes an acetic acid buffer and chitosan hydrochloride having a deacetylation of greater than 85% and a viscosity of 1 cp to 200 cp. In some embodiments, the chitosan adjuvant includes an acetic acid buffer and chitosan hydrochloride having a deacetylation of greater than 75% and a viscosity of 1 cp to 200 cp. In some embodiments, the chitosan adjuvant includes an acetic acid buffer and chitosan hydrochloride having a deacetylation of greater than 85% and a viscosity of 5 cp to 100 cp. In some embodiments, the chitosan adjuvant includes a sodium chloride buffer and chitosan hydrochloride having a deacetylation of greater than 75% and a viscosity of 1 cp to 200 cp. In some embodiments, the chitosan adjuvant includes a sodium chloride buffer and chitosan hydrochloride having a deacetylation of greater than 85% and a viscosity of 5 cp to 100 cp.

In some embodiments, the chitosan adjuvant includes an acid-soluble chitosan and a buffer. In some embodiments, the chitosan adjuvant includes a buffer and an acid-soluble chitosan having a deacetylation of greater than 75% and a viscosity of 1 cp to 200 cp. In some embodiments, the chitosan adjuvant includes an acetic acid buffer and an acid-soluble chitosan having a deacetylation of greater than 75% and a viscosity of 1 cp to 200 cp. In some embodiments, the chitosan adjuvant includes an acetic acid buffer and an acid-soluble chitosan having a deacetylation of greater than 85% and a viscosity of 1 cp to 200 cp. In some embodiments, the chitosan adjuvant includes an acetic acid buffer and an acid-soluble chitosan having a deacetylation of greater than 85% and a viscosity of 5 cp to 100 cp. In some embodiments, the chitosan adjuvant includes an acid-soluble chitosan and a histidine buffer. In some embodiments, the chitosan adjuvant includes a buffer and an acid-soluble chitosan having a deacetylation of greater than 75% and a viscosity of 1 cp to 200 cp. In some embodiments, the chitosan adjuvant includes a sodium chloride buffer and an acid-soluble chitosan having a deacetylation of greater than 75% and a viscosity of 1 cp to 200 cp. In some embodiments, the chitosan adjuvant includes a sodium chloride buffer and an acid-soluble chitosan having a deacetylation of greater than 85% and a viscosity of 5 cp to 100 cp.

In some embodiments, the chitosan adjuvant includes a tonicity modifier. In some embodiments, the tonicity modifier is selected from any pharmaceutically acceptable tonicity modifiers, such as sodium chloride, potassium chloride, sucrose, trehalose and combinations thereof. In some embodiments the tonicity modifiers is present in the amount of 10 mM to 500 mM.

In some embodiments, the chitosan adjuvant includes a water-soluble chitosan and a tonicity modifier. In some embodiments, the chitosan adjuvant includes an acid-soluble chitosan and a tonicity modifier. In some embodiments, the chitosan adjuvant includes chitosan hydrochloride and a tonicity modifier. In some embodiments, the chitosan adjuvant includes a tonicity modifier and a water-soluble chitosan having a deacetylation of greater than 75% and a viscosity of 1 cp to 200 cp. In some embodiments, the chitosan adjuvant includes a tonicity modifier and an acid-soluble chitosan having a deacetylation of greater than 75% and a viscosity of 1 cp to 200 cp. In some embodiments, the chitosan adjuvant includes a sodium chloride and water-soluble chitosan having a deacetylation of greater than 85% and a viscosity of 5 cp to 100 cp. In some embodiments, the chitosan adjuvant includes a sodium chloride and an acid-soluble chitosan having a deacetylation of greater than 85% and a viscosity of 5 cp to 100 cp.

In some embodiments, the chitosan adjuvant includes a detergent. In some embodiments, the detergent is selected from any pharmaceutically acceptable detergents, such as Polysorbate 80, Polysorbate 20, Poloxamer 188, and combinations thereof. In some embodiments the detergents are present in the amount of 0.001 to 0.2% (w/v).

In some embodiments, the chitosan adjuvant includes a water-soluble chitosan and a detergent. In some embodiments, the chitosan adjuvant includes an acid-soluble chitosan and a detergent. In some embodiments, the chitosan adjuvant includes chitosan hydrochloride and a detergent. In some embodiments, the chitosan adjuvant includes a detergent and a water-soluble chitosan having a deacetylation of greater than 75% and a viscosity of 1 cp to 200 cp. In some embodiments, the chitosan adjuvant includes a detergent and an acid-soluble chitosan having a deacetylation of greater than 75% and a viscosity of 1 cp to 200 cp.

### The HPV VLP-Based Vaccine

Any HPV VLP-based vaccine is suitable for use in the pharmaceutical compositions and methods of the present invention. Known HPV VLP vaccines can be modified to include both an aluminum adjuvant and a PLGA. New vaccines can be developed according to the invention described herein that comprise at least one HPV type, optionally in the form of an HPV VLP adsorbed to an aluminum adjuvant, in combination with PLGA. Additionally, new vaccines can be developed according to the invention described herein that comprise at least one HPV type in the form of an HPV VLP adsorbed to an aluminum adjuvant in combination PLGA.

One exemplary HPV vaccine is a bivalent vaccine protective against HPV 16 and 18, which is known commercially as CERVARIX® (GlaxoSmithKline Biologicals, Rixensart, Belgium). Another exemplary HPV VLP vaccine is a non-infectious recombinant, quadrivalent vaccine prepared from highly purified VLPs of the major capsid (L1) protein of HPV types 6, 11, 16, and 18, and may be referred to herein by its proprietary name GARDASIL® (Merck & Co., Inc., Rahway, NJ, USA), see Bryan, J. T. Vaccine 25(16): 3001-6 (2007); Shi et al. Clinical Pharmacology and Therapeutics 81(2): 259-64 (2007). Another exemplary HPV VLP vaccine is the nine-valent vaccine marketed for prevention of HPV (that includes the capsid (L1) protein of HPV types 6, 11, 16, 18, 31, 33, 45, 52, and 58), which is referred to herein by its proprietary name GARDASIL®9 (Merck & Co., Inc., Rahway, NJ, USA).

In some embodiments, the vaccine dose includes, in addition to VLPs, an aluminum adjuvant (as amorphous aluminum hydroxyphosphate sulfate), sodium chloride, L-histidine, polysorbate 80, sodium borate, and water. In some embodiments, the HPV vaccine includes 100-3500 μg aluminum adjuvant, 1-50 mg sodium chloride, 0.05-10 mg L-histidine, 1-100 μg polysorbate, 1-100 μg sodium borate, and water. In some embodiments, the HPV vaccine includes about 500 μg aluminum adjuvant, about 9.56 mg sodium chloride, about 0.78 mg L-histidine, about 50 μg polysorbate 80, about 35 μg sodium borate, and water for injection. Known HPV VLP vaccines can be modified to include both an aluminum adjuvant and PLGA in accordance with the present invention.

In some embodiments of the invention, the pharmaceutical compositions and formulations comprise HPV VLP-based vaccines, or HPV VLPs as described herein, that are monovalent, bivalent, trivalent, quadrivalent, 5-valent, 6-valent, 7-valent, 8-valent or 9-valent. In particular embodiments, the pharmaceutical compositions and formulations are 9-valent. In some embodiments, the pharmaceutical compositions comprise HPV VLP-based vaccines, or HPV VLPs as described herein, with more than four different types of HPV VLPs. For example, the pharmaceutical compositions and formulations of the invention may include HPV VLP-based vaccines, or HPV VLPs as described herein, that are 8-valent, 9-valent, 10-valent, and so forth. For example, pharmaceutical compositions comprising VLPs of HPV 16 and/or HPV 18, without the inclusion of other HPV VLP types, are included within the scope of the invention. Multi-valent vaccines comprising different HPV VLPs than the HPV types included in GARDASIL® or GARDASIL®9 are also contemplated herein.

In some embodiments, the VLPs of HPV types 6 and 11 are included. In some embodiments, the VLPs of HPV types 16, 31, and 35 are included. In some embodiments, the VLPs of HPV types 18, 45, and 59 are included. In some embodiments, the VLPs of HPV types 26, 51, and 69 are included. In some embodiments, the VLPs of HPV types 33, 52, and 58 are included. In some embodiments, the VLPs of HPV types 39, 68, and 70 are included. In some embodiments, the VLPs of HPV types 53, 56, and 66 are included.

In some embodiments, the VLPs of HPV types 16 and 18 are included. In some embodiments, the VLPs of HPV types 6, 11, 16, and 18 are included. In some embodiments, the VLPs of HPV types 6, 18, 52, and 58 are included. In some embodiments, the VLPs of HPV types 6, 11, 16, 18, 31, 45, 52, and 58 are included. In some embodiments, the VLPs of HPV types 6, 11, 16, 18, 33, 45, 52, and 58 are included. In some embodiments, the VLPs of HPV types 6, 11, 16, 18, 31, 33, 45, 52, and 58 are included. In some embodiments, the VLPs of HPV types 6, 11, 16, 18, 31, 33, 45, 52, and 59 are included. In some embodiments, the VLPs of HPV types 6, 11, 16, 18, 31, 33, 45, 53, and 58 are included. In some embodiments, the VLPs of HPV types 6, 11, 16, 18, 31, 33, 45, 53, and 59 are included. In some embodiments, the VLPs of HPV types 6, 11, 16, 18, 31, 33, 35, 45, 52, and 58 are included. In some embodiments, the VLPs of HPV types 6, 11, 16, 18, 31, 33, 35, 45, 52, 58, and 59 are included. In some embodiments, the VLPs of HPV types 6, 11, 16, 18, 31, 33, 45, 52, 58, 59, and 68 are included. In some embodiments, the VLPs of HPV types 6, 11, 16, 18, 31, 33, 35, 39, 45, 51, 52, 56, 58, and 59 are included. In some embodiments, the VLPs of HPV types 6, 11, 16, 18, 26, 31, 33, 35, 45, 51, 52, 58, 59, and 69 are included. In some embodiments, the VLPs of HPV types 6, 11, 16, 18, 26, 31, 33, 35, 39, 45, 51, 52, 58, 59, 68, 69, and 70 are included. In some embodiments, the VLPs of HPV types 6, 11, 16, 18, 26, 31, 33, 35, 39, 45, 51, 52, 53, 56, 58, 59, 66, 68, 69, and 70 are included.

In some embodiments, the pharmaceutical compositions and formulations comprise HPV VLP-based vaccines and/or antigens as listed in Table 1 below:

TABLE 1

| Name | Antigen | Adjuvant | Party |
|---|---|---|---|
| CERVARIX ® | L1 VLP of HPV-16 and | Aluminum | GlaxoSmithKline |
| (2vHPV vaccine) | HPV-18 | hydroxide and | Biologics (Rixensart, |
| | | MPL | Belgium) |
| GARDASIL ® | L1 VLP of HPV-6, HPV-11, | AHSS | Merck & Co., Inc., |
| (4vHPV vaccine) | HPV-16 and HPV-18 | | Rahway NJ USA |
| GARDASIL ® 9 | L1 VLP of HPV-6, HPV-11, | AHSS | Merck& Co., Inc., |
| (9vHPV vaccine) | HPV-16, HPV-18, HPV-31, | | Rahway NJ USA |
| | HPV-33, HPV-45, HPV-52 | | |
| | and HPV-58 | | |
| CECOLIN ® | L1 VLP of HPV-16 and | Aluminum | Xiamen Innovax |
| | HPV-18 | hydroxide | |
| GEOCOLIN ® | L1 VLP of HPV-6 and | Aluminum | Xiamen Innovax |
| | HPV-11 | hydroxide | |
| L1 capsomers | L1 capsomers of HPV-16 | unknown | R. Garcea, University |
| | | | of Colorado- Boulder |
| RG1-VLP | HPV-16 L1-L2 (17-36) VLP | Aluminum | R. Kirnbauer, NCI, |
| | | hydroxide | Pathovax LLC |
| L2-AAV | L2 peptides of HPV-16 and | unknown | 2A Pharma |
| | HPV-31 displayed on AAV | | |
| | VLP | | |
| L2 multimer | Fusion protein of L2 ~11-88 | Alum | Sanofi, BravoVax |
| | of HPV-6, HPV-16, HPV-18, | | |
| | HPV-31 and HPV-39 | | |
| L2-thioredoxin | L2 peptide displayed on | unknown | M. Muller, DKFZ |
| | thioredoxin | | |
| AX03 | L2 peptide displayed on | unknown | Agilvax, NIAID |
| | bacteriophage | | |
| L1-E7 VLP | HPV-16 L1-E7 VLP | None | Medigene AG |
| TA-CIN | HPV-16 L2E7E6 fusion | None | Cantab |
| | protein | | Pharmaceuticals, |
| | | | Xenova |
| TA-GW | HPV-6 L2E7 fusion protein | Aluminum | Cantab |
| | | hydroxide or | Pharmaceuticals, GSK |
| | | AS03 | |

In some embodiments, the composition of the present invention is administered as a single injection that provides multiple-doses of the composition.

In some embodiments, the composition includes a buffer. In some embodiments, the composition includes a buffer, where the buffer is selected from the group consisting of: acetic acid, histidine, citrate, Bis-Tris, HEPES, phosphate, MES, sodium chloride, and combinations thereof.

In some embodiments, the composition includes a salt.

In some embodiments, the composition includes a tonicity modifier. In some embodiments, the tonicity modifier is selected from the group consisting of sodium chloride, potassium chloride, sucrose, trehalose and combinations thereof.

In some embodiments, the composition includes a detergent. In some embodiments, the detergent is selected from the group consisting of Polysorbate 80, Polysorbate 20, Poloxamer 188, and combinations thereof.

### Injectable Compositions

Formulations of the present invention are suitable for parenteral administration, such as, for example, by intraarticular (in the joints), intravenous or intraperitoneal injection, and preferably by intramuscular, intradermal or subcutaneous injection, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials. Injection solutions and suspensions can be prepared from sterile powders, granules, and tablets. Cells transduced by the RNA molecules can also be administered intravenously or parenterally.

The compositions of the present invention are suitable for injection through a needle into a host. In one embodiment, the compositions comprise microparticles suspended in an aqueous injection vehicle. The microparticles preferably have a mass median diameter of at least about 10 μm to about 250 μm, preferably in the range of from about 20 μm to about 150 μm. However, it should be understood that the invention is not limited to microparticles in this size range, and that smaller or larger microparticles may also be used.

A controlled release composition of the present invention may provide the following advantages: 1) it provides immediate release after entering into a body without drug release lag phase, in both high or low drug-loading; 2) it is conducive to scaled-up production (scale above 75 L) during the production; 3) it is highly stable, and therefore in vivo release behavior will not change after long-term storage.

### Kits of the Invention

Also provided herein are kits including any of the pharmaceutical compositions of vaccines as described above and instructions for use.

Also provided herein are kits including a pharmaceutical composition comprising HPV VLPs of at least one type of HPV.

In some embodiments of the kits, the pharmaceutical composition of (a) comprises HPV VLPs of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82. In some embodiment, the pharmaceutical composition of (a) is an HPV vaccine. In some embodiments, the HPV vaccine is a Human Papillomavirus Bivalent (Types 16 and 18) Vaccine, Recombinant. In some embodiments, the HPV vaccine is CERVARIX®. In some embodiments, the HPV vaccine is a Human Papillomavirus Quadrivalent (Types 6, 11, 16, 18) Vaccine, Recombinant. In some embodiments, the HPV vaccine is GARDASIL®. In some embodiments, the HPV vaccine is a Papillomavirus 9-valent Vaccine, Recombinant. In some embodiments, the HPV vaccine is GARDASIL®9.

In some embodiments, the kit includes a buffer. In some embodiments, the kit includes a tonicity modifier. In some embodiments, the kit includes a detergent.

In some embodiments of the kits, the kit includes a label or packaging insert that includes a description of the components and/or instructions for use in vivo of the components therein. In some embodiments, the kits include instructions for administering (or vaccinating) the pharmaceutical composition or HPV Vaccine. In some embodiments, the kits include instructions for admixing the pharmaceutical composition or HPV vaccine and subsequentially administering (or vaccinating) the admixture to a patient.

### Methods of Treatment of the Invention

Also provided herein is a method of inducing an immune response to a human papillomavirus (HPV) in a human patient comprising administering to the patient a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of inducing an immune response to a human papillomavirus (HPV) in a human patient including administering a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of inducing an immune response to a human papillomavirus (HPV) in a human patient including administering to the patient a pharmaceutical composition comprising a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of preventing infection of a human patient by a human papillomavirus (HPV) including administration to the patient a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of delivering a pharmaceutical composition to a host that induces a neutralizing titer against an HPV antigen in the host that includes administering to the host a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, whereby the administration of the pharmaceutical composition induces a neutralizing titer against the HPV antigen in the host, and wherein a single dose of the pharmaceutical composition provides enhanced or comparable neutralizing titers when compared to multiple doses of the same pharmaceutical composition.

Also provided herein is a method for preventing cancer of a human patient cancers caused by human papillomavirus (HPV) Types 16, 18, 31, 33, 45, 52, and 58 including administration to the patient a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is cervical, vulvar, vaginal, anal, oropharyngeal, and other head and neck cancers.

Also provided herein is a method for preventing cancer of a human patient caused by HPV Types 6, 11, 16, 18, 31, 33, 45, 52, and 58 including administration to the patient a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is cervical, vulvar, vaginal, and anal precancerous or dysplastic lesions.

Also provided herein is a method for preventing cancer of a human patient caused by HPV Types 6 and 11 including administration to the patient a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is genital warts or condyloma acuminata.

Also provided herein is a method for preventing precancerous or dysplastic lesions of a human patient caused by HPV Types 6, 11, 16, 18, 26, 31, 33, 35, 39, 45, 51, 52, 53, 55, 56, 58, 59, 66, 68, 73, and 82 including administration to the patient a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the lesions are selected from Cervical intraepithelial neoplasia (CIN) grade 2/3, cervical adenocarcinoma in situ (AIS), Cervical intraepithelial neoplasia (CIN) grade 1, Vulvar intraepithelial neoplasia (VIN) grade 2 and grade 3, Vaginal intraepithelial neoplasia (VaIN) grade 2 and grade 3, Anal intraepithelial neoplasia (AlN) grades 1, 2, and 3. (1.1).

Also provided herein is a method for preventing HPV-related anogenital disease of a human patient caused by HPV Types selected from the group consisting of HPV types: 6, 11, 16, 18, 26, 31, 33, 35, 39, 45, 51, 52, 53, 55, 56, 58, 59, 66, 68, 73, and 82 including administration to the patient a pharmaceutical composition including a polymer (e.g. poly(lactide-co-glycolide)) and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Embodiments of the invention also include one or more of the pharmaceutical compositions described herein (i) for use in, (ii) for use as a medicament or composition for, or (iii) for use in the preparation of a medicament for: (a) therapy (e.g., of the human body); (b) medicine; (c) induction of an immune response against HPV types included in the vaccine (d) decreasing the likelihood of HPV infection in a patient; (e) prevention of infection with HPV types in the vaccine, (f) prevention or reduction of the likelihood of cervical cancer, (g) prevention or reduction of the likelihood of vulvar cancer, (h) prevention or reduction of the likelihood of vaginal cancer, (i) prevention or reduction of the likelihood of anal cancer, (j) prevention or reduction of the likelihood of oropharyngeal cancer, (k) prevention or reduction of the likelihood of other head and neck cancers, (k) prevention or reduction of the likelihood of precancerous or dysplastic anal lesions, (1) prevention or reduction of the likelihood of genital warts or condyloma acuminata, (m) prevention or reduction of the likelihood of Cervical intraepithelial neoplasia (CIN) grade 2/3 lesions, (n) prevention or reduction of the likelihood of cervical adenocarcinoma in situ (AIS) lesions, (o) prevention or reduction of the likelihood of Cervical intraepithelial neoplasia (CIN) grade 1 lesions, (p) prevention or reduction of the likelihood of Vulvar intraepithelial neoplasia (VIN) grade 2 and grade 3 lesions, (q) prevention or reduction of the likelihood of Vaginal intraepithelial neoplasia (VaIN) grade 2 and grade 3 lesions, (r) prevention or reduction of the likelihood of Anal intraepithelial neoplasia (AlN) grades 1, 2, and 3 lesions.

In embodiment 1, a pharmaceutical composition is provided comprising:
virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82;
a polymer comprising poly(lactide-co-glycolide); and
a pharmaceutically acceptable carrier.

In embodiment 2, the pharmaceutical composition of embodiment 1 is provided, wherein the VLPs are present in an amount of 0.01-20 wt % of the composition.

In embodiment 3, the pharmaceutical composition of embodiment 1 is provided, wherein the polymer is present in an amount 80-99.09 wt % of the composition.

In embodiment 4, the pharmaceutical composition suitable for parenteral administration is provided, comprising:
(a) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82;
(b) a polymer comprising poly(lactide-co-glycolide); and
(c) a pharmaceutically acceptable carrier,
wherein the weight content of the HVP vaccine component in the composition is within a range from 0.01% to 20%; the weight content of the polymer in the pharmaceutical composition is within a range from 80% to 99.09%; and
wherein the polymer has an intrinsic viscosity of 0.4-0.9 dl/g, and a molar ratio of lactide to glycolide of 65:35 to 90:10.

In embodiment 5, the pharmaceutical composition of any of embodiment 1-4 is provided, wherein the poly(lactide-co-glycolide) is an ester capped poly(lactide-co-glycolide) having a molar ratio of lactide to glycolide 50:50.

In embodiment 6, the pharmaceutical composition of any of embodiment 1-5 is provided, wherein the HPV VLPs of each of the at least one HPV types are present in a concentration of about 10 μg to about 300 μg per 0.5 mL of the pharmaceutical composition.

In embodiment 7, the pharmaceutical composition of any of embodiment 1-6, is provided wherein the total VLP concentration is between 10 μg and 2000 μg per 0.5 mL of the pharmaceutical composition.

In embodiment 8, the pharmaceutical composition of any of embodiment 1-7 is provided, wherein the total poly(lactide-co-glycolide)concentration is between 0.1 μg to about 200 mg per 0.5 mL of the pharmaceutical composition.

In embodiment 9, the pharmaceutical composition of any of embodiment 1-8 is provided, further comprising an adjuvant.

In embodiment 10, the pharmaceutical composition of any of embodiment 1-9 is provided, further comprising about 100 μg to about 3500 μg of an aluminum adjuvant.

In embodiment 11, the pharmaceutical composition of embodiment 10 is provided, wherein the HPV VLPs are adsorbed onto the aluminum adjuvant.

In embodiment 12, the composition of any of embodiment 1-11 is provided, wherein each of the HPV VLPs are present in a concentration of about 10 μg to about 300 μg per 0.5 mL of the pharmaceutical composition; wherein the total HPV VLP concentration is between 10 μg and 2000 μg per 0.5 mL of the pharmaceutical composition; and wherein the polymer has an intrinsic viscosity of 0.4-0.9 dl/g, a molar ratio of lactide to glycolide of 65:35 to 90:10.

In embodiment 13, the pharmaceutical composition of any of embodiment 1-2 is provided wherein the HPV VLPs comprise HPV L1 protein and do not comprise HPV L2 protein.

In embodiment 14, the pharmaceutical composition of any of embodiment 1-13 is provided, wherein the VLPs are associated with the poly(lactide-co-glycolide).

In embodiment 15, the pharmaceutical composition of any of embodiment 1-13 is provided, wherein the VLPs are encapsulated in the poly(lactide-co-glycolide).

In embodiment 16, the pharmaceutical composition of any of embodiment 1-13 is provided, wherein the VLPs are embedded in the poly(lactide-co-glycolide).

In embodiment 17, the pharmaceutical composition of any of embodiment 1-16 is provided, wherein the composition is in the form of microparticles.

In embodiment 18, the pharmaceutical composition of any of embodiment 1-16 is provided, wherein the composition is in the form of microspheres.

In embodiment 19, the pharmaceutical composition of any of embodiment 1-16 is provided, wherein the composition is in the form of a minitab.

In embodiment 20, the pharmaceutical composition of any of embodiment 1-19 is provided, wherein the composition is administered as a single injection that provides multiple-doses of the VLP over a period of time.

In embodiment 21, a method of preventing infection of a human patient by a human papillomavirus (HPV) is provided comprising administering to the patient the pharmaceutical composition of any of embodiments 1-20.

In embodiment 22, a method of delivering a pharmaceutical composition to a host is provided that induces a neutralizing titer against an HPV antigen in the host comprising: administering to the host the pharmaceutical composition of any of embodiments 1-20.

In embodiment 23, a method is provided of forming a biodegradable implant in situ in a patient in need thereof comprising the steps of
injecting the composition of any of embodiments 1-19 into the body of the patient.

In embodiment 24, a kit is provided comprising:
(a) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82; a poly(lactide-co-glycolide); and a pharmaceutically acceptable carrier; and
(b) a delivery device.

In embodiment 25, the kit of embodiment 24 is provided, further comprising instructions for administering to a human patient the HPV vaccine and the poly(lactide-co-glycolide).

In embodiment 26, a pulsatile drug delivery system is provided comprising:
a microneedle assembly including a plurality of microneedles filled with a composition comprising:
(a) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82;
(b) poly(lactide-co-glycolide); and
(c) a pharmaceutically acceptable carrier,
   wherein the microneedle assembly is configured to release the VLPs at predetermined times with a predetermined amount of the VLPs while the microneedle assembly remains embedded in a patient.
(d) In embodiment 27, the delivery system of embodiment 26 is provided, wherein the PLGA degrades over time to release the VLPs into the patient.

### EXAMPLES

The following examples are meant to be illustrative and should not be construed as further limiting. The contents of the figures and all references, patents, and published patent applications cited throughout this application are expressly incorporated herein by reference.

### Example 1: Spray Drying of HPV/AAHS

A formulation comprising of 0.54 mg/mL HPV VLPs, 8.7 mg/mL AAHS, 9.5% trehalose, 0.5% trileucine, 75 mM NaCl, 30 mM histidine, 0.01% PS-80, pH 6.2 was spray dried using two spray dryers, the ProCept and the Buchi B-290. The parameters are listed in Table 2 below.

TABLE 2
Spray dryer conditions for ProCept and Buchi processes.

| | | |
|---|---|---|
| Process Variabl | ProCepT Setpoint | Buchi Setpoint |

| | | | | |
|---|---|---|---|---|
| Inlet T | 143° | C. | 102° | C. |
| Outlet T | 57° | C. | 60° | C. |
| Bifluid Nozzle ID | 1 | mm | 0.7 | mm |
| Drying Air Flow Rate | 0.3 | m³/min | 600 | L/min |
| Atomization Air Flow Rate | 8.5 | L/min | 9 | L/min |
| Solution Pump Rate | 3.3 | g/min | 3.5 | g/min |

The temperature used in the described process include an inlet temperature of 143 C for the ProCept unit and 102 C for the Buchi unit, well above the melt temperature of the VLPs which is 59-61 C. The spray dried material was transferred to glass vials, stoppered and placed on stability at 37 C for 0, 2 and 4 weeks. Samples were analyzed for potency using the Biacore 3000 and for AAHS particle size by static light scattering. Analysis showed that the AAHS particle size and HPV potency were stable for up to 4 weeks at 37 C. Moisture content was 4.6% and 5.7% for the Buchi and ProCept units, respectively. FIG. 3 depicts an SEM image of ProCept spray dried material. FIGS. 4A and 4B depicts the characterization of spray dried material at 0. 2 and 4 weeks at 37 C in sealed containers. Specifically, FIG. 4A shows the potency by Biacore analysis compared to a reference standard. FIG. 4B depicts particle size of AAHS by static light scattering.

### Example 2: Temperature Programmed Loading of HPV to PLGA Microparticles

There are three FDA approved prophylactic HPV vaccines: Cervarix™, Gardasil®, and Gardasil®9. The most recent HPV vaccination recommended by the Advisory Committee on Immunization Practices (ACIP) remains as a 2-dose or 3-dose schedule to get full protection from new HPV infections. However, immunization non-compliance occurs when people fail to complete the dose series and this remains as a public health issue. To improve vaccine coverage and adherence with the immunization schedule, a long-acting HPV formulation with controlled release of HPV that could be given in combination with the current market product Gardasil®9 during one single doctor visit was prepared.

The successful encapsulation of HPV virus-like particles (VLP, ˜20,000 kDa) was achieved with blank PLGA microparticles containing adjuvant aluminum hydroxyphosphate sulfate (AAHS, aka AAHS) by active loading of HPV VLPs.

As shown in the schematic of FIG. 5, microparticles were prepared as follows. First, AAHS concentrated with 4% trehalose was prepared. 10 mL AAHS stock solution in FAP buffer (500 mM NaCl, 20 mM histidine, 0.02 wt % PS80, pH 6.2) was transferred into 15 mL falcon tube and 0.87 mL 50% trehalose solution was added and mixed. The inner water phase was prepared by centrifuging AAHS solution with trehalose at 1500 rpm for 15 min at 15° C. and removal of all supernatant. 0.2 mL AAHS water phase was added to 1 mL of the polymer oil phase, then homogenized at 8 k rpm for 60s with IKA dispenser (T25 Easy Clean Digital equipped with S 25 N-8 G dispersing tool). To the resulting primary w/o emulsion, 2 mL 5% PVA (9-10 kDa, 80% hydrolyzed) was added and vortexed at 2750 rpm for 60 s. In the end, the final w/o/w emulsion was added into 100 mL 0.5% PVA solution and microparticles were hardened under rapid stirring for a minimal of 3 h. The microparticles were collected and fractioned by passing through 106 μm, 53 um, and 20 um sequentially and rinsed repeatedly with DI water to remove residual PVA. Particles were collected and lyophilized followed by Gamma irradiation at ˜19-20 kGy for sterilization.

To prepare sterile blank AAHS/PLGA microparticles, 38-54 kDa PLGA (Resomer® RG 504, ester terminated, L:G ratio 50:50) was dissolved in 1 mL dichloromethane to achieve a concentration of 300 mg/mL. All formulations, as shown in FIG. 6, were prepared with 100% 38-54 kDa PLGA and the following specifications, as set forth in Table 3. Formulation A was homogenized at 8000 rpm for 60 s, vortex at 3000 rpm for 60 s. Formulation B was homogenized at 5000 rpm for 45 s, vortex at 1500 rpm for 30 s. Formulation C was homogenized at 6000 rpm for 45 s, vortex at 1500 rpm for 35 s. Formulation D was homogenized at 15000 rpm for 60 s, vortexed at 3000 rpm for 60 s.

TABLE 3

| | Polymer | 38-54 kDa | | Vortex |
|---|---|---|---|---|
| | conc. | PLGA | Homogenization | speed |
| Formulation | (mg/mL) | wt % | rate (rpm) | (rpm) |
| A | 300 | 100 | 8000 | 3000 |
| B | 300 | 100 | 5000 | 1500 |
| C | 300 | 100 | 6000 | 1500 |
| D | 300 | 100 | 15000 | 3000 |

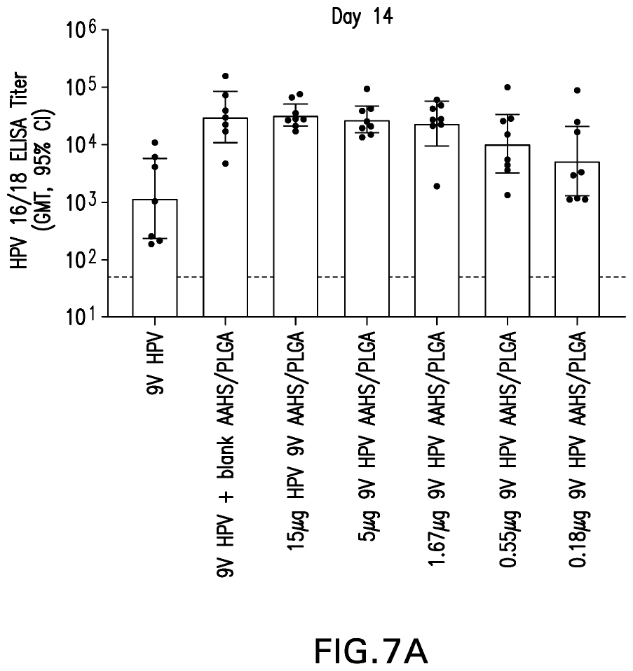

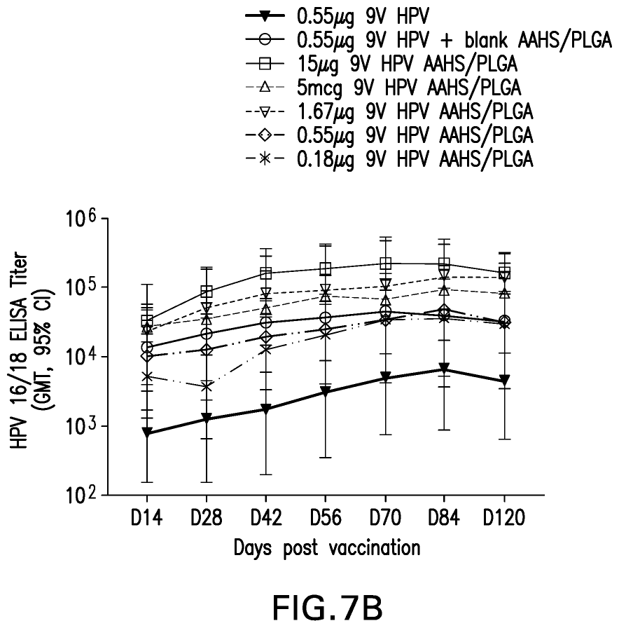

The potency of HPV33 post-encapsulation was largely retained (>80%) as shown in FIG. 7. Additionally, the active loading enabled by strong binding affinity of HPV to AAHS in blank PLGA microparticles led to a high encapsulation efficiency (>85%). This provides advantages such as reducing cost associated with the use of excessive high-value, high-cost drug substance in the loading process when compared to traditional encapsulation techniques.

Sterile 9V HPV/AAHS/PLGA microparticles were prepared as shown in FIG. 5 and described in detail above. The formulations were diluted with buffer containing carboxymethylcellulose, sodium chloride, histidine, polysorbate 80 to achieve different dose levels. The formulations were tested in BALB/c mice model with 8 mice per group using a single dose of microparticle from 0.18 μg to 15 μg dose, as shown in FIG. 7A. To assess immunogenicity, samples of sera from individual animals were evaluated using a multiplex assay for antibody levels to HPV type 16 and 18. VLP-specific HPV antibody concentrations were determined at study day 14, 28, 42, 56, 70, 84, 120. Titers to HPV VLP-16 and HPV VLP-18 are shown in FIG. 7B. The HPV16/18 released from formulation were immunogenic and successfully elicited HPV16/18 titer in a dose-dependent manner.

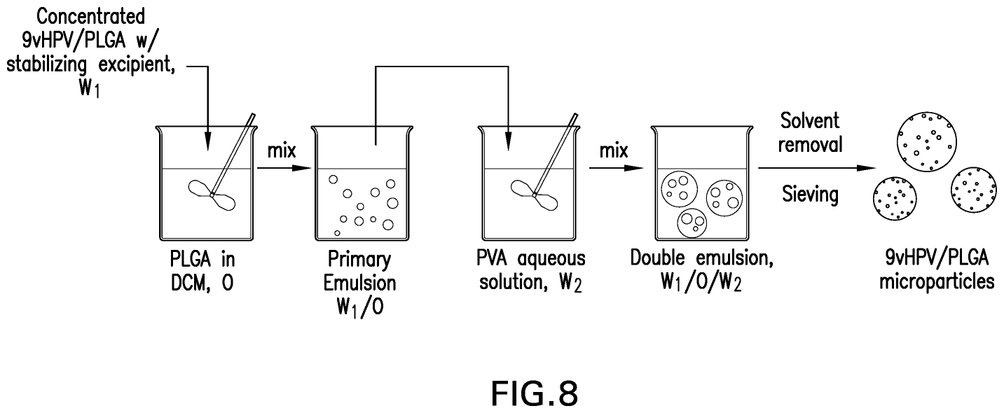

The stability of HPV through emulsification process was significantly enhanced when used as bound form with adjuvant AAHS. Thus HPV/AAHS (e.g., G9) was successfully encapsulated in PLGA microparticles with a w/o/w double emulsion process as shown in FIG. 8. When used in conjunction of G9, G9/PLGA microparticles and 9V HPV/AAHS/PLGA microparticles prepared via temperature-programmed loading process demonstrated non-inferior performance when compared to benchmark G9 prime and boost (day 28) in rabbit model.

### Example 3: Delayed Release Minitab

Minitabs according to the present invention were made by a compaction method. The tablets can incorporate vaccines selected from subunit, conjugated and/or live virus (LVV) in adjuvanted and unadjuvanted form (i.e. Virus-like particles (VLPs) and aluminum hydroxyphosphate sulfate (AAHS)) in a range of dimensions and coated with a variety of polymers for either injection or surgical implantation to delay or prolong the release of the antigen. Compaction of materials into tablets require pressures in the range of 50 MPa to 400 MPa. These stresses occurring during compaction can damage proteins and due to the large size of many vaccines they are more vulnerable to these types of stresses than smaller proteins or small molecule pharmaceuticals. The formulation and binder combination are important to allow for adequate compressibility and compactability. Once compacted, the minitablet is coated by dropping the minitabs in a compatible anti-solvent containing the polymer of choice.

In this example, a 9 valent HPV/aluminum adjuvant vaccine that included the capsid (L1) protein of HPV types 6, 11, 16, 18, 31, 33, 45, 52, and 58 (hereinafter “9vHPV Vaccine”) was tested (the 9V vaccine further included sodium chloride, L-histidine, and polysorbate 80, and, where noted also contained aluminum (provided as AAHS)). The nine-valent HPV types 6, 11, 16, 18, 31, 33, 45, 52 and 58, were diluted into a buffer with the final concentration of 5.0 wt % sucrose, 4.5 wt % trehalose, 3.0 wt % HPMC, 10 mM histidine, 75 mM NaCl, 0.01 v/v % PS80 with and without approximately 2 wt % AAHS. In addition, a lyophilized formulation was prepared as above without HPV or AAHS adjuvant as a placebo formulation. The three formulations, HPV no AAHS, HPV+AAHS and placebo were dispensed into 2 r tubing vials, at 0.5 mL per vial, Igloo stopper was added half-seated, and then vials were placed on the lyophilizer shelf and lyophilized. The HPV+AAHS was prepared as set forth in Table 4 below.

TABLE 4

| | Liquid concentration | |
|---|---|---|
| | (mg/mL) | % Mass |
| 9vHPV | 0.135 | 0.100 |
| AAHS | 2.18 | 1.63 |
| Sucrose | 50.0 | 37.4 |
| Trehalose | 45.2 | 33.9 |
| NaCl | 4.38 | 3.28 |
| Histidine | 1.56 | 1.16 |
| HPMC | 30.0 | 22.5 |
| PS80 | 0.100 | 0.070 |
| Total | 133 | 100 |

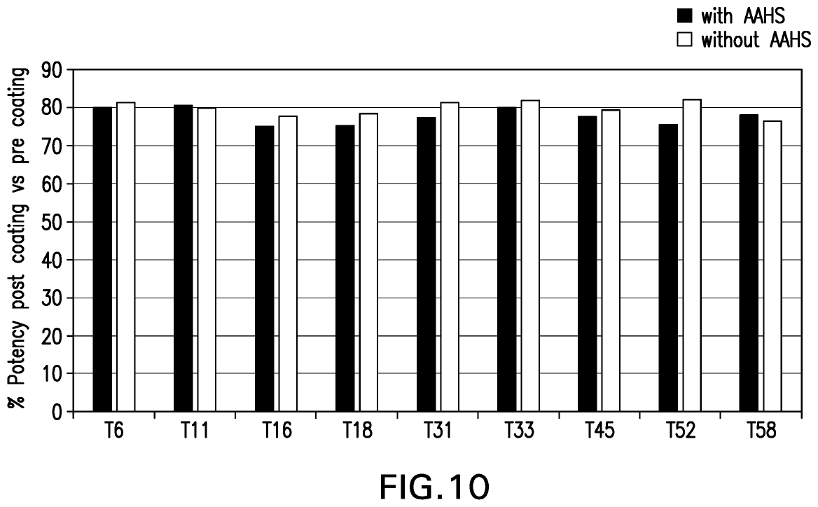

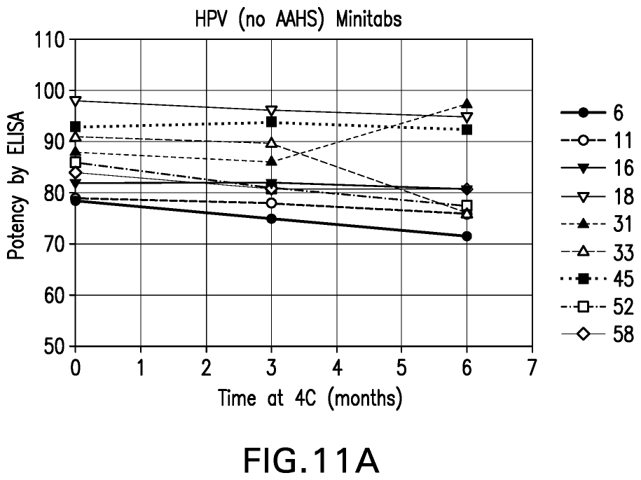

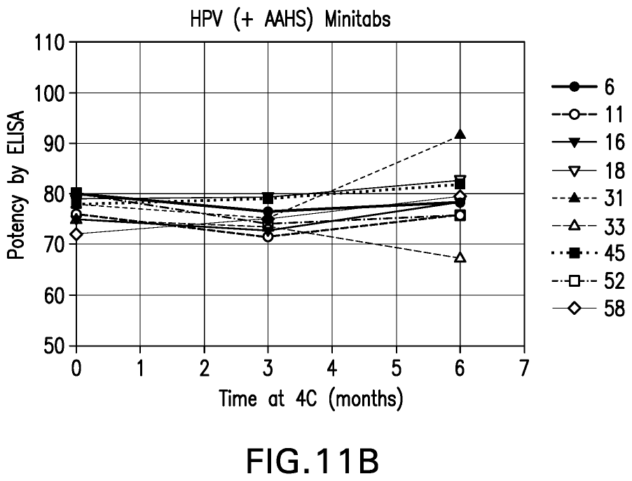

At the end of the cycle, the vials were stoppered and removed from the lyophilizer. Lyophilized HPV was combined from multiple vials, milled by passing through a sieve, blended with binders (lactose, mannitol) and then lubricated with magnesium stearate with the following final composition: 1% lyophilized formulation, 30% mannitol, 59% lactose, 1% magnesium stearate. This material was compacted using 2 mm diameter round punches with curvature to a compaction pressure of approximately 125 MPa. Minitabs were coated by submerging and agitating the minitabs in a 20% solution of 85%/15% PLGA in acetone. Coating thickness can be modulated by repeating the coating process, The resulting minitabs were approximately 2×2 millimeters. The potency of all 9 HPV types post coating was similar for all types in both the minitabs with AAHS and those without AAHS, as shown in FIG. 10. Further, it was found that all 9 HPV types were stable over 6 months at 4 C in formulations that did not include AAHS (see FIG. 11A) and those that did include AAHS (see FIG. 11B).

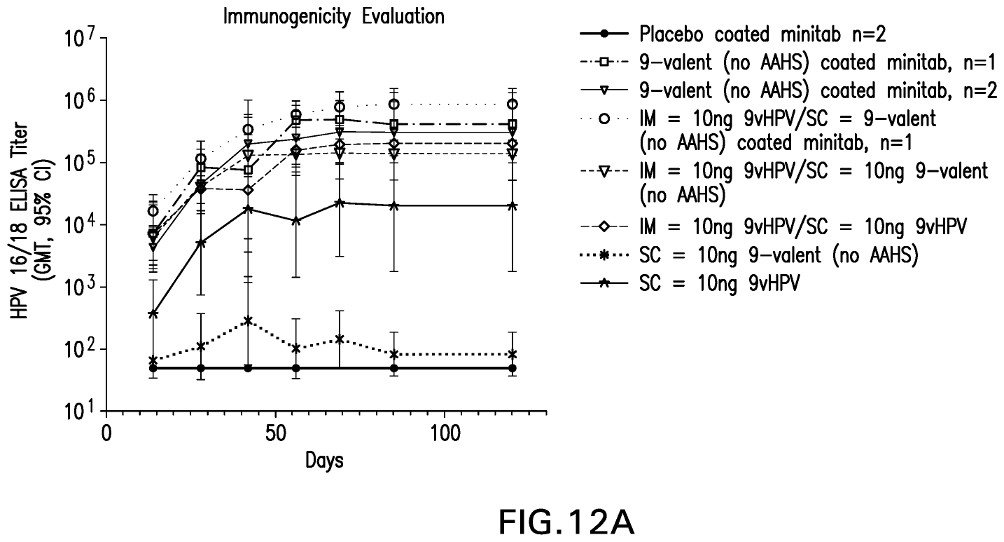

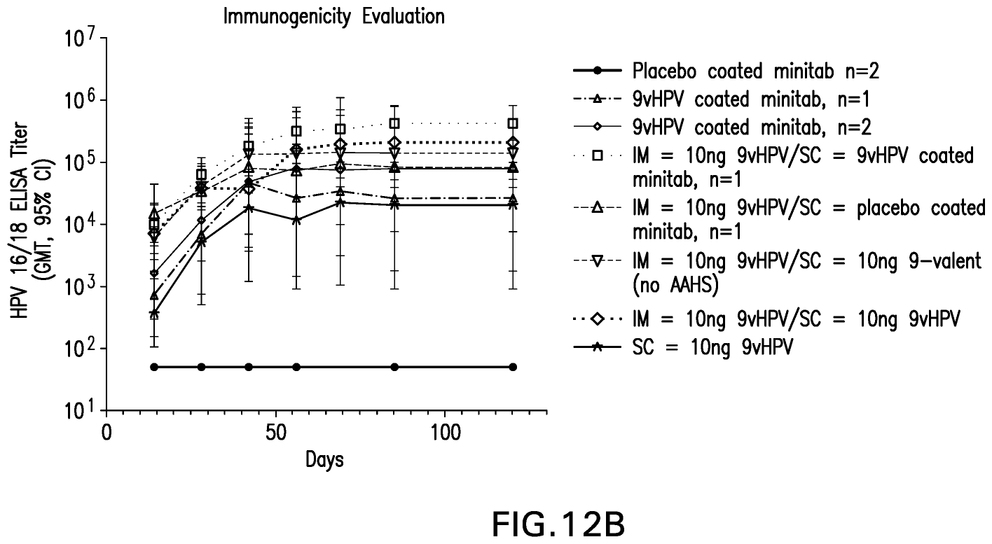

A mouse immunogenicity study was designed to evaluate the potency of these formulations compared to the benchmark 9VHPV mouse dose. 12 different formulations were tested, as outlined in Table 5 below. As shown from the immunogenicity evaluation in FIGS. 12A and 12B, intramuscular ELISA titers for both 9vHPV and 9vHPV+AAHS provided significant improvement over the placebo. In contrast, subcutaneous 9vHPV+AAHS performed better than both placebo and subcutaneous 9vHPV, without AAHS.

TABLE 5

| | Number | | | | |
|---|---|---|---|---|---|
| | of | Prime | Prime | Boost (# | Boost |
| Group | Mice | Inoculum | ROA | mintabs) | ROA |
| 1 | 8 | N/A | N/A | Placebo | SC, surgical |
| | | | | minitab (2) | implantation |
| 2 | 8 | N/A | N/A | 9vHPV coated | SC, surgical |
| | | | | minitab (1) | implantation |
| 3 | 8 | N/A | N/A | 9vHPV + AAHS | SC, surgical |
| | | | | coated minitab | implantation |
| | | | | (1) | |
| 4 | 8 | N/A | N/A | 9vHPV coated | SC, surgical |
| | | | | minitab (1) | implantation |
| 5 | 8 | N/A | N/A | 9vHPV + AAHS | SC, surgical |
| | | | | coated minitab | implantation |
| | | | | (2) | |
| 6 | 8 | 9vHPV + | IM | 9vHPV coated | SC, surgical |
| | | AAHS | | minitab (1) | implantation |
| 7 | 8 | 9vHPV + | IM | 9vHPV coated | SC, surgical |
| | | AAHS | | minitab (1) | implantation |
| 8 | 8 | 9vHPV + | IM | Placebo | SC, surgical |
| | | AAHS | | minitab | implantation |
| 9 | 8 | 9vHPV + | IM | 9vHPV | SC, liquid |
| | | AAHS | | | injection |
| 10 | 8 | 9vHPV + | IM | 9vHPV | SC, liquid |
| | | AAHS | | | injection |
| 11 | 8 | 9vHPV | SC | N/A | N/A |
| 12 | 8 | 9vHPV + | SC | N/A | N/A |
| | | AAHS | | | |

IM = intramuscular;
NA = not applicable;
SC = subcutaneous

### Example 4: Delayed Release Vaccine Implant Including VLP, in Presence and Absence of an Adjuvant

The process includes the preparation of dried vaccine intermediate to <5% moisture using lyophilization, spray drying, spray freeze drying or microwave vacuum drying. This dried vaccine intermediate is then sieved or milled (if necessary) to a free-flowing powder. This powder is added to a polymer or polymer blend and extruded through a twin-screw extruder at a controlled rate and temperature.

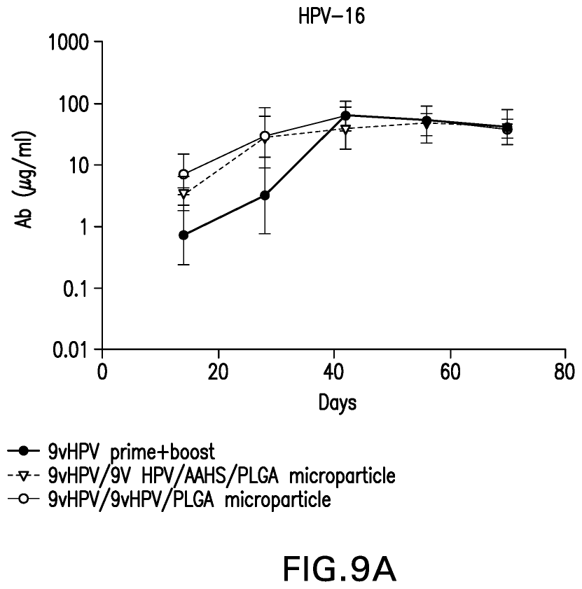

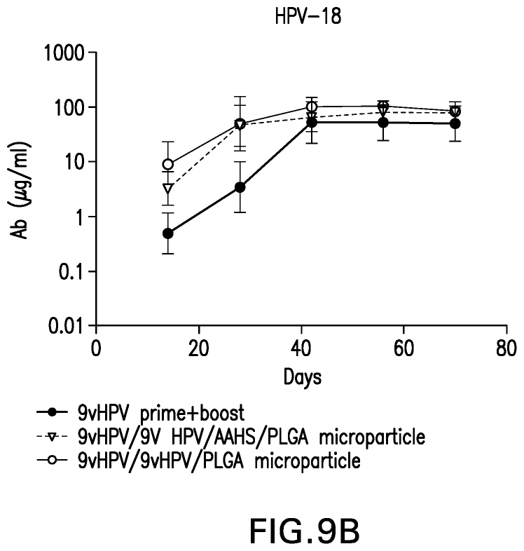

In the example, HPV33 VLPs were lyophilized in the presence of 6.3% sucrose, 6.3% trehalose, 6.3% HPMC, 113 mM NaCl, 12 mM histidine and 0.012% PS90 and dried to a moisture <5%. The lyophilized intermediate was crushed into a powder. The extrusion conditions are listed in Table 6 below. A total of 0.6 mg was used to prime extruder and confirm suitable parameters (ratio of 1:1 HPV33 intermediate to PEG3350) and was discarded. An additional 2 g HPV33 intermediate+2 g of PEG3350 was extruded. See FIG. 9.

TABLE 6
Extruder parameters
7.5 mm Extruder: FDC-059 w/ 2 mm die

| Motor rpm | 125 | |
|---|---|---|
| Max torque | 40% | |
| Feed rate | 30% | |
| Zone 1 | 65° | C. |
| Zone 2 | 75° | C. |
| Zone 3 | 85° | C. |

| | |
|---|---|
| Zone 4 | off |

The extrudate was cut into segments for analysis. Protein concentration, HPV33 particle size and potency were evaluated. The extrudate was weighed and reconstituted in 1 mL 0.5M NaCl, 20 mM histidine, 0.02% PS-80, pH 6.2.

For two reconstituted extruded pieces, the protein concentration was 495 and 330 μg/mL, respectively. Particle size of the HPV33 was also measured after diluting with 0.5M NaCl, 20 mM histidine, 0.02% PS-80, pH 6.2, and was reconstituted to reduce the concentration of PEG in the sample. Measurement was performed on a Malvern Nanosizer instrument. Controls of HPV33 alone and HPV33 spiked with the same PEG level as the samples were also measured.

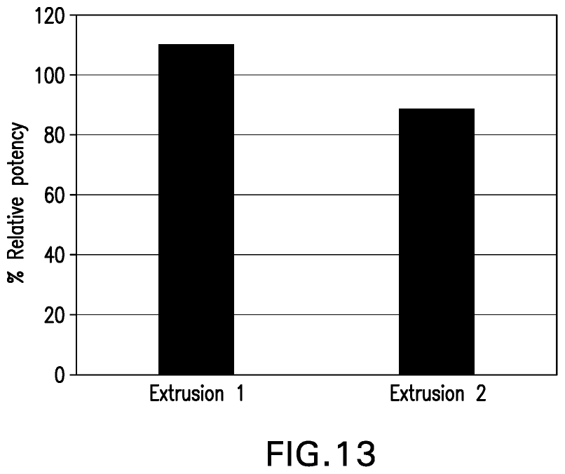

Potency measured by SPR analysis compared to a HPV33 reference standard was 110% and 89%, respectively See FIG. 13.

TABLE 7
Particle size measurement by DLS
of reconstituted extruded product

| Sample name | Particle size (d · nm) |
|---|---|
| HPV alone | 60.97 |
| HPV PEG spike in | 72.55 |
| Extrusion sample 1 | 71.51 |
| Extrusion sample 2 | 67.57 |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A pharmaceutical composition comprising:
virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82;
a polymer comprising poly(lactide-co-glycolide); and
a pharmaceutically acceptable carrier.

2. The pharmaceutical composition of claim 1, wherein the VLPs are present in an amount of 0.01-20 wt % of the composition.

3. The pharmaceutical composition of claim 1, wherein the polymer is present in an amount 80-99.09 wt % of the composition.

4. A pharmaceutical composition suitable for parenteral administration, comprising:
(a) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82;
(b) a polymer comprising poly(lactide-co-glycolide); and
(c) a pharmaceutically acceptable carrier,
wherein the weight content of the VLPs in the composition is within a range from 0.01% to 20%; the weight content of the polymer in the pharmaceutical composition is within a range from 80% to 99.09%; and
wherein the polymer has an intrinsic viscosity of 0.4-0.9 dl/g, and a molar ratio of lactide to glycolide of 65:35 to 90:10.

5. The pharmaceutical composition of claim 4, wherein the poly(lactide-co-glycolide) is an ester capped poly(lactide-co-glycolide) having a molar ratio of lactide to glycolide 50:50.

6. The pharmaceutical composition of claim 4, wherein the HPV VLPs of each of the at least one HPV types are present in a concentration of about 10 μg to about 300 μg per 0.5 mL of the pharmaceutical composition.

7. The pharmaceutical composition of claim 6, wherein the total VLP concentration is between 10 μg and 2000 μg per 0.5 mL of the pharmaceutical composition.

8. The pharmaceutical composition of claim 4, wherein the total poly(lactide-co-glycolide)concentration is between 0.1 μg to about 200 mg per 0.5 mL of the pharmaceutical composition.

9. The pharmaceutical composition of claim 4, further comprising an adjuvant.

10. The pharmaceutical composition of claim 9, further comprising about 100 μg to about 3500 μg of an aluminum adjuvant.

11. The pharmaceutical composition of claim 10, wherein the HPV VLPs are adsorbed onto the aluminum adjuvant.

12. The composition of claim 4, wherein each of the HPV VLPs are present in a concentration of about 10 μg to about 300 μg per 0.5 mL of the pharmaceutical composition; wherein the total HPV VLP concentration is between 10 μg and 2000 μg per 0.5 mL of the pharmaceutical composition; and wherein the polymer has an intrinsic viscosity of 0.4-0.9 dl/g, a molar ratio of lactide to glycolide of 65:35 to 90:10.

13. The pharmaceutical composition of claim 4, wherein the HPV VLPs comprise HPV L1 protein and do not comprise HPV L2 protein.

14. The pharmaceutical composition of claim 4, wherein the VLPs are associated with the poly(lactide-co-glycolide).

15. The pharmaceutical composition of claim 4, wherein the VLPs are encapsulated in the poly(lactide-co-glycolide).

16. The pharmaceutical composition of claim 4, wherein the VLPs are embedded in the poly(lactide-co-glycolide).

17. The pharmaceutical composition of claim 4, wherein the composition is in the form of microparticles.

18. The pharmaceutical composition of claim 4, wherein the composition is in the form of microspheres.

19. The pharmaceutical composition of claim 4, wherein the composition is administered as a single injection that provides multiple-doses of the VLPs over a period of time.

20. A method of preventing infection of a human patient by a human papillomavirus (HPV) comprising administering to the patient the pharmaceutical composition of claim 1.

21. A method of delivering a pharmaceutical composition to a host that induces a neutralizing titer against an HPV antigen in the host comprising:
administering to the host the pharmaceutical composition of claim 1.

22. A method of forming a biodegradable implant in situ in a patient in need thereof comprising the steps of:
injecting the composition of claim 1 into the body of the patient.

23. A kit comprising:
(a) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82; a poly(lactide-co-glycolide); and a pharmaceutically acceptable carrier; and
(b) a delivery device.

24. The kit of claim 23, further comprising instructions for administering to a human patient the HPV vaccine and the poly(lactide-co-glycolide).

25. A pulsatile drug delivery system comprising:
a microneedle assembly including a plurality of microneedles filled with a composition comprising:
(a) virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 69, 70, 73, and 82;
(b) poly(lactide-co-glycolide) (“PLGA”); and
(c) a pharmaceutically acceptable carrier,
wherein the microneedle assembly is configured to release the VLPs at predetermined times with a predetermined amount of the VLPs while the microneedle assembly remains embedded in a patient.

26. The delivery system of claim 25, wherein the PLGA degrades over time to release the VLPs into the patient.
